(19) United States
(12) Patent Application Publication
(10) Pub. No.: US 2023/0414628 A1
(43) Pub. Date: Dec. 28, 2023

(54) METHODS FOR TREATING DISEASES MEDIATED BY GLUTATHIONE PEROXIDASE 4

(71) Applicant: Sanath Kumar Ramesh, San Jose, CA (US)

(72) Inventor: Sanath Kumar Ramesh, San Jose, CA (US)

(21) Appl. No.: 18/339,816

(22) Filed: Jun. 22, 2023

Related U.S. Application Data
(60) Provisional application No. 63/355,506, filed on Jun. 24, 2022.

(51) Int. Cl. A61K 31/5377; A61K 31/4422; A61K 31/496; A61K 31/55; A61K 31/4535; A61K 31/519; A61K 31/085; A61K 31/05; A61K 31/122; A61K 31/381; A61K 31/352; A61K 31/57; A61K 31/567; A61K 31/09; A61K 31/36; A61K 31/24; A61K 31/403; A61K 31/4704; A61K 31/4174; A61K 31/4985; A61K 31/5513; A61K 31/485; A61K 31/5415; A61K 31/136; A61K 31/437; A61K 31/506; A61K 31/517; A61K 31/4745; A61K 31/47; A61K 31/404; A61K 31/473; A61K 31/415; A61K 31/137; A61K 31/4375; A61K 31/353; A61K 31/135; A61K 31/495; A61K 31/4439; A61K 31/472; A61K 31/4035; A61K 31/4709; A61K 31/551; A61K 31/426; A61K 31/444; A61K 31/416; A61K 31/15; A61K 31/4178; A61K 31/216; A61K 31/175; A61K 31/40; A61K 31/4164

(52) U.S. Cl. CPC A61K 31/5377; A61K 31/4422; A61K 31/496; A61K 31/55; A61K 31/4535; A61K 31/519; A61K 31/085; A61K 31/05; A61K 31/122; A61K 31/381; A61K 31/352; A61K 31/57; A61K 31/567; A61K 31/09; A61K 31/36; A61K 31/24; A61K 31/403; A61K 31/4704; A61K 31/4174; A61K 31/4985; A61K 31/5513; A61K 31/485; A61K 31/5415; A61K 31/136; A61K 31/437; A61K 31/506; A61K 31/517; A61K 31/4745; A61K 31/47; A61K 31/404; A61K 31/473; A61K 31/415; A61K 31/137; A61K 31/4375; A61K 31/353; A61K 31/135; A61K 31/495; A61K 31/4439; A61K 31/472; A61K 31/4035; A61K 31/4709; A61K 31/551; A61K 31/426; A61K 31/444; A61K 31/416; A61K 31/15; A61K 31/4178; A61K 31/216; A61K 31/175; A61K 31/40; A61K 31/4164

(57) ABSTRACT

The present disclosure relates generally to compounds and pharmaceutical compositions for treating or preventing diseases, disorders, or conditions mediated by glutathione peroxidase 4 (GPX4) and methods of use thereof.

### CROSS-REFERENCE TO RELATED APPLICATIONS

This application claims the benefit under 35 U.S.C. §119(e) of U.S. Provisional Application No. 63/355,506, filed Jun. 24, 2022, which is hereby incorporated by reference in its entirety.

### FIELD

Provided herein are compounds and pharmaceutical compositions for treating or preventing diseases, disorders, or conditions mediated by glutathione peroxidase 4 (GPX4) and methods of use thereof.

### BACKGROUND

Sedaghatian type Spondylometaphyseal Dysplasia (SSMD) is an extremely rare progressive disorder in which ends of the long bones (metaphyses) show cupping/flaring, flattening of the vertebrae (platyspondyly), cardiac arrhythmia, and central nervous system abnormalities including hypogenesis of corpus callosum and cerebellar hypoplasia. The disease is congenital, and the majority of patients die in the first days after birth due to respiratory distress. SSMD is an autosomal recessive disease caused by variants in both alleles of the glutathione peroxidase 4 (GPX4) gene. GPX4 is a member of the family of antioxidant defense enzymes called glutathione peroxidases, and protects the cells against membrane lipid peroxidation. Whole exome sequencing of a child affected with SSMD (and unaffected parents) revealed that mutations in GPX4 are likely responsible for SSMD. A small number of patient GPX4 sequences have been reported, and include both point mutations and missense mutations.

Based on natural history data of known patients, other symptoms include severe hypotonia, global development delays, auditory neuropathy, cortical visual impairment, scoliosis, and hypertonia. The oldest patient developed intractable seizures at the age of 3 and continues to be treated with anticonvulsants to reduce the occurrence of breakthrough seizures. There is no current therapy to treat SSMD, except for physical and occupational therapies. Without treatment, babies born with this condition can never sit up, walk, have feeding difficulties, and have significantly delayed physical and cognitive development. They are at a high risk for premature death by cardiovascular, cerebrovascular, neuromuscular, or renal complications. Thus, there exists a need for treatments for SSMD.

### SUMMARY

The present disclosure provides methods for treating a disease, disorder, or condition mediated by glutathione peroxidase 4 (GPX4) in a human patient in need thereof, comprising administering to the human patient an effective amount of a compound or a pharmaceutically acceptable salt or solvate thereof, wherein the compound is selected from the group consisting of a calcium channel blocker, radical trapping antioxidant (RTA), contraceptive, phytoestrogen antioxidant, antipsychotic, dopaminergic, serotonergic, iron chelator, opioid receptor antagonist, δ receptor antagonist, neuromodulator, S1P₃ receptor antagonist, Cdc25 phosphatase inhibitor, antimalarial, PPARdelta antagonist, NAMPT activator, autophagy activator, neurogenesis activator, smoothened agonist, STAT3 inhibitor, antiviral, LDHA inhibitor, PDGFRbeta receptor inhibitor, NAT10 inhibitor, FXR agonist, CFTR potentiator, S1P2 receptor antagonist, GPR139 agonist, HDAC class II inhibitor, multidrug resistance pump inhibitor, and gamma secretase inhibitor.

Also provided herein are methods for treating a disease, disorder, or condition mediated by glutathione peroxidase 4 (GPX4) in a human patient in need thereof, comprising administering to the human patient an effective amount of a compound of Table 1A or 1B, or a pharmaceutically acceptable salt thereof.

Also provided herein are methods for treating a disease, disorder, or condition mediated by glutathione peroxidase 4 (GPX4) in a human patient in need thereof, comprising administering to the human patient an effective amount of a compound of Table 1A, or a pharmaceutically acceptable salt thereof.

Also provided herein are methods for treating a disease, disorder, or condition mediated by glutathione peroxidase 4 (GPX4) in a human patient in need thereof, comprising administering to the human patient an effective amount of a dihydropyridine calcium channel blocker or a pharmaceutically acceptable salt thereof.

Also provided herein are methods for treating a disease, disorder, or condition mediated by glutathione peroxidase 4 (GPX4) in a human patient in need thereof, comprising administering to the human patient an effective amount of azelnidipine or a pharmaceutically acceptable salt thereof.

Also provided herein are methods for treating a disease, disorder, or condition mediated by glutathione peroxidase 4 (GPX4) in a human patient in need thereof, comprising administering to the human patient an effective amount of bazedoxifene or a pharmaceutically acceptable salt thereof.

Also provided herein are methods for treating a disease, disorder, or condition mediated by glutathione peroxidase 4 (GPX4) in a human patient in need thereof, comprising administering to the human patient an effective amount of estrogen receptor modulator.

Also provided herein are methods for treating a disease, disorder, or condition mediated by glutathione peroxidase 4 (GPX4) in a human patient in need thereof, comprising administering to the human patient a pharmaceutical composition comprising an effective amount of a compound described herein and a pharmaceutically acceptable carrier.

Also provided herein are methods for treating a disease, disorder, or condition mediated by glutathione peroxidase 4 (GPX4) in a human patient in need thereof, comprising administering to the human patient a pharmaceutical composition comprising an effective amount of dihydropyridine calcium channel blocker or a pharmaceutically acceptable salt thereof and a pharmaceutically acceptable carrier.

Also provided herein are methods for treating a disease, disorder, or condition mediated by glutathione peroxidase 4 (GPX4) in a human patient in need thereof, comprising administering to the human patient a pharmaceutical composition comprising an effective amount of azelnidipine or a pharmaceutically acceptable salt thereof and a pharmaceutically acceptable carrier.

Also provided herein are methods for treating a disease, disorder, or condition mediated by glutathione peroxidase 4 (GPX4) in a human patient in need thereof, comprising administering to the human patient a pharmaceutical composition comprising an effective amount of bazedoxifene or a pharmaceutically acceptable salt thereof and a pharmaceutically acceptable carrier.

Also provided herein are methods for treating a disease, disorder, or condition mediated by glutathione peroxidase 4 (GPX4) in a human patient in need thereof, comprising administering to the human patient a pharmaceutical composition comprising an effective amount of free radical trapping antioxidant and a pharmaceutically acceptable carrier.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.05%. Also, to the term “about X” includes description of “X.”

Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Provided are also pharmaceutically acceptable salts, stereoisomers, mixture of stereoisomers, hydrates, solvates, solid forms, and tautomeric forms of the compounds described herein.

In many cases, the compounds of this disclosure are capable of forming acid and/or base salts by virtue of the presence of amino and/or carboxyl groups or groups similar thereto.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salt” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “solvate” refers to a complex formed by a combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc.

Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “solid form” refers to a type of solid-state material that includes amorphous as well as crystalline forms. The term “crystalline form” refers to polymorphs as well as solvates, hydrates, etc. The term “polymorph” refers to a particular crystal structure having particular physical properties such as X-ray diffraction, melting point, and the like.

Some of the compounds exist as “tautomers.” Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Likewise, some of the compounds exist as “stereoisomers.” The term stereoisomer refers to molecules where covalently bonded atoms have the same atomic connectivity but some of the atoms have different spatial orientation from each other. For example, compounds may exist as optical stereoisomers. Stereoisomers each contain one or more chiral centers, and may exist in two or more stereoisomeric forms referred to as enantiomers or diastereomers. For another example, compounds may exist as geometric isomers, e.g. cis- or trans-isomers, where orientation of substituents on adjacent carbons of a double bond differ between the stereoisomers. Thus, compounds described herein may be single stereoisomers (for example, essentially free of other stereoisomers), racemates, or mixtures of enantiomers or diastereomers. All such single stereoisomers, racemates, and mixtures thereof are contemplated within the scope of the present invention.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³ H (tritium), ¹³C, ¹³C, ¹⁴C, ¹⁵N, ¹⁸F, ³¹P, ³²P, ³⁵S, ³⁶Cl, and ¹²⁵I. Various isotopically labeled compounds of the present disclosure include, for example, those into which radioactive isotopes such as ³H, ¹³C, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound described herein when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out syntheses known in the art and substituting a readily available isotopically labeled reagent for a non-isotopically labeled reagent.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” or “patient” refers to an animal, such as a mammal (including a human), that has been or will be the object of treatment, observation or experiment. The methods described herein may be useful in human therapy and/or veterinary applications. In some embodiments, the subject or patient is a mammal In some embodiments, the subject or patient is a human.

The term “therapeutically effective amount” or “effective amount” of a compound described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a condition or disorder described herein, including but not limited to GPX4 deficiency. The therapeutically effective amount may vary depending on the subject, disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Compounds, Pharmaceutical Compositions, and Modes of Administration

Provided herein are compounds useful for treating or preventing a disease, disorder, or condition mediated by glutathione peroxidase 4 (GPX4) in a human patient in need thereof Also provided herein are compounds useful for treating or preventing a disease, disorder, or condition associated with a deficiency in GPX4. Also provided herein are compounds useful for upregulating the expression of GPX4.

In some embodiments, the disease, disorder, or condition is associated with a deficiency in GPX4. In some embodiments, the disease, disorder, or condition being treated or prevented is Sedaghatian-type Spondylometaphyseal Dysplasia (SSMD). In some embodiments, the disease, disorder, or condition being treated or prevented is a neurodegenerative disease. In some embodiments, the disease, disorder or condition being treated or prevented is a mitochondrial disease. In some embodiments, the disease, disorder, or condition being treated or prevented is associated with ferroptosis. In some embodiments, the disease, disorder, or condition being treated or prevented is an ischemic heart disease. In some embodiments, the disease, disorder, or condition being treated or prevented is male infertility.

Provded herein are methods for treating a disease, disorder, or condition mediated by glutathione peroxidase 4 (GPX4) in a human patient in need thereof, comprising administering to the human patient an effective amount of a compound or a pharmaceutically acceptable salt thereof, wherein the compound is selected from the group consisting of a calcium channel blocker, radical trapping antioxidant (RTA), contraceptive, phytoestrogen antioxidant, antipsychotic, dopaminergic, serotonergic, iron chelator, opioid receptor antagonist, a δ receptor antagonist, neuromodulator, S1P₃ receptor antagonist, Cdc25 phosphatase inhibitor, antimalarial, PPARdelta antagonist, NAMPT activator, autophagy activator, neurogenesis activator, smoothened agonist, STAT3 inhibitor, antiviral, LDHA inhibitor, PDGFRbeta receptor inhibitor, NAT10 inhibitor, FXR agonist, CFTR potentiator, S1P₂ receptor antagonist, GPR139 agonist, HDAC class II inhibitor, multidrug resistance pump inhibitor, and gamma secretase inhibitor.

Provded herein are methods for treating SSMD in a human patient in need thereof, comprising administering to the human patient an effective amount of a compound or a pharmaceutically acceptable salt thereof, wherein the compound is selected from the group consisting of a calcium channel blocker, radical trapping antioxidant (RTA), contraceptive, phytoestrogen antioxidant, antipsychotic, dopaminergic, serotonergic, iron chelator, opioid receptor antagonist, a δ receptor antagonist, neuromodulator, S1P₃ receptor antagonist, Cdc25 phosphatase inhibitor, antimalarial, PPARdelta antagonist, NAMPT activator, autophagy activator, neurogenesis activator, smoothened agonist, STAT3 inhibitor, antiviral, LDHA inhibitor, PDGFRbeta receptor inhibitor, NAT10 inhibitor, FXR agonist, CFTR potentiator, S1P₂ receptor antagonist, GPR139 agonist, HDAC class II inhibitor, multidrug resistance pump inhibitor, and gamma secretase inhibitor.

In some embodiments, the compound is a calcium channel blocker. The term “calcium channel blocker” refers to an agent that can disrupt the movement of calcium ions through calcium channels. Calcium channel blockers can be used as an antihypertensive agent. In some embodiments, the compound is an antihypertensive calcium channel blocker. Calcium channel blockers include, but are not limited to, dihydropyridines (such as manidipine, azelnidpine, nifedipine and amlodipine, and can be referred to as dihydropyridine calcium channel blockers) and non-dihydropyridine (such as verapamil and diltiazem).

In some embodiments, the calcium channel blocker is manidipine, or a pharmaceutically acceptable salt, tautomer, stereoisomer, deuterated analog, or solvate thereof

In some embodiments, the compound is a GABA-ergic. “GABA” refers to γ-aminobutyric acid, which is the main inhibitory neurotransmitter in the adult mammalian central nervous system (CNS). “GABAergic” or “GABA-ergic” refers to an agent that modulates GABA.

GABA mediates its effect through metabotropic GABA_{B} receptors as well as ionotrophic GABA_{A} and GABA_{C} receptors. GABA_{B} receptors are present both on presynaptic terminals and postsynaptic neurons involved in fine-tuning of several neurotransmitter systems. In some embodiments, the GABA-ergic is a GABA_{B} modulator. GABA_{B} modulators may be GABA_{B} receptor activating compounds, such as positive allosteric modulators of the GABA_{B} receptor.

In some embodimetns, the compound is GABAB modulator. In some embodiments, the GABAB modulator is CGP-7930 (2,6-ditert-butyl-4-(3-hydroxy-2,2-dimethylpropyl)phenol) or a pharmaceutically acceptable salt, tautomer, stereoisomer, deuterated analog, or solvate thereof. In some embodiments, the GABA B modulator is CGP-13501 (3-(3,5-Ditert-butyl-4-hydroxyphenyl)-2,2-dimethylpropanal) or a pharmaceutically acceptable salt, tautomer, stereoisomer, deuterated analog, or solvate of each thereof.

In some embodiments, the compound is an antibiotic. An antibiotic is a class of compounds that fights bacterial infections. In some embodiments, the antibiotic is rifaximin or a pharmaceutically acceptable salt, tautomer, stereoisomer, deuterated analog, or solvate thereof. In some embodiments, the antibiotic is clofoctol or a pharmaceutically acceptable salt, tautomer, stereoisomer, deuterated analog, or solvate thereof.

In some embodiments, the compound is a radical trapping antioxidant (RTA). Radicals, or free radicals, are highly energetic chemical species that have a tendency to non-discriminatorily react with substrates thereby causing damages thereon. RTAs are a class of compounds capable of capturing the radicals, thereby protecting sensitive substrates or mitigating the aforementioned damages. RTAs are sometimes referred to as free radical trapping antioxidants.

In some embodiments, the compound is a cryptic RTA.

In some embodiments, the cryptic RTA is an estrogen receptor modulator. Estrogen receptor (ER) is a group of proteins that are receptors activated by the hormone estrogen. Activated ER may translocate into the nucleus and bind to DNA to regulate the expression of many estrogen responsive target genes, or alternatively assert other functions independent of DNA binding. In some embodiments, the cryptic RTA is an ER agonist. ER agonist binds to ER and triggers estrogen signaling pathways in the target cells, much like the estrogen itself. In some embodiments, the ER agonist is SNG1153 or a pharmaceutically acceptable salt, tautomer, stereoisomer, deuterated analog, or solvate thereof.

In some embodiments, the cryptic RTA is an ER antagonist. An ER antagonist blocks ER and inhibits transcription by promoting the binding of co-repressors, thereby inhibiting estrogen actions. In some embodiments, the ER antagonist is ZK-164015 or G-15, or a pharmaceutically acceptable salt, tautomer, stereoisomer, deuterated analog, or solvate of each thereof

In some embodiments, the cryptic RTA is a selective estrogen receptor modulator (SERM). SERM is also known as ER agonist/antagonist. It is a class of compounds that acts on ER, but differs from “pure” ER agonists and antagonists in that its action differs in different tissues, allowing for selective inhibition or stimulation of estrogen-like actions in different tissues.

In some embodiments, the compound is a selective estrogen receptor modulator (SERM). In some embodiments, the SERM is Y-134, raloxifene, or a pharmaceutically acceptable salt, tautomer, stereoisomer, deuterated analog, or solvate of each thereof. In some embodiments, the SERM is bazedoxifene or a pharmaceutically acceptable salt, tautomer, stereoisomer, deuterated analog, or solvate thereof.

In some embodiments, the cryptic RTA is a beta-adrenergic. A beta-adrenergic is a sympathomimetic agent acting upon a beta-adrenergic receptor. In some embodiments, the beta-adrenergic is a beta-adrenergic blocker, which is a competitive antagonist that blocks all or some of the beta-adrenergic receptor sites.

In some embodiments, the compound is beta-adrenergic blocker. In some embodiments, the beta-adrenergic blocker is metipranolol, carazolol, or carvedilol, or a pharmaceutically acceptable salt, tautomer, stereoisomer, deuterated analog, or solvate of each thereof. In some embodiments, the beta-adrenergic is a beta-adrenergic agonist, which activates some or all of the beta-adrenergic receptor sites. In some embodiments, the compound is a beta-adrenergic agonist. In some embodiments, the beta-adrenergic agonist is indacaterol or a pharmaceutically acceptable salt, tautomer, stereoisomer, deuterated analog, or solvate thereof.

In some embodiments, the cryptic RTA is an alpha-adrenergic. An alpha-adrenergic is a sympathomimetic agent acting upon an alpha-adrenergic receptor.

In some embodiments, the alpha-adrenergic is an alpha-antagonist, which is a competitive antagonist that blocks all or some of the alpha-adrenergic receptor sites. In some embodiments, the alpha-antagonist is moxisylyte or a pharmaceutically acceptable salt, tautomer, stereoisomer, deuterated analog, or solvate thereof. In some embodiments, the alpha-adrenergic is an alpha-agonist, which activates some or all of the alpha-adrenergic receptor sites. In some embodiments, the alpha-agonist is oxymetazoline or a pharmaceutically acceptable salt, tautomer, stereoisomer, deuterated analog, or solvate thereof. In some embodiments, the compound is alpha-adrenergic. In some embodiments, the compound is alpha-adrenergic antagonist. In some embodiments, the compound is alpha-adrenergic agonist.

In some embodiments, the RTA is an anti-inflammatory. In some embodiments, the compound is an anti-inflammatory. In some embodiments, the anti-inflammatory is a LOX/COX inhibitor. A LOX/COX inhibitor refers to a dual inhibitor of lipoxygenase (LOX) and cyclooxygenase (COX). In some embodiments, the LOX/COX inhibitor is FPL 62064, or a pharmaceutically acceptable salt, tautomer, stereoisomer, deuterated analog, or solvate thereof

In some embodiments, the compound is LOX/COX inhibitor, leukotriene production blocker, antioxidant, or 5-lipoxygenase inhibitor.

In some embodiments, the anti-inflammatory is a leukotriene production blocker. Leukotriene is an inflammatory promotor synthesized in response to, for example, receptor activation, antigen-antibody interaction, and physical stimuli, such as cold temperatures. A leukotriene production blocker inhibits the production of leukotriene by, for example, blocking 5-lipoxygenase activity. In some embodiments, the leukotriene production blocker is a tryptanthrin or a pharmaceutically acceptable salt, tautomer, stereoisomer, deuterated analog, or solvate thereof.

In some embodiments, the anti-inflammatory is a 5-lipoxygenase inhibitor. A 5-Lipoxygenase inhibitor blocks the activity of 5-lipoxygenase, thereby inhibiting the production of leukotriene. In some embodiments, the 5-lipoxygenase inhibitor is zileuton or a pharmaceutically acceptable salt, tautomer, stereoisomer, deuterated analog, or solvate thereof.

In some embodiments, the anti-inflammatory is an antioxidant. In some embodiments, the antioxidant is butylated hydroxyanisole, honokiol, menadione, idebenone, or LY 231617, or a pharmaceutically acceptable salt, tautomer, stereoisomer, deuterated analog, or solvate of each thereof.

In some embodiments, the compound is a contraceptive. In some embodiments, the contraceptive is mifepristone, ulipristal acetate, or ethinyl estradiol, or a pharmaceutically acceptable salt, tautomer, stereoisomer, deuterated analog, or solvate of each thereof.

In some embodiments, the compound is a phytoestrogen antioxidant. Phytoestrogens are a class of biphenolic compounds that can bind to estrogen receptors and assert estrogen or antiestrogen effect.

Phytoestrogen can function as an antioxidant due to the phenolic functional groups, and thus is also referred to as phytoestrogen antioxidant. In some embodiments, the phytoestrogen antioxidant is licochalcone, resveratrol, pterostilbene, nordihydroguaiaretic acid, or macelignan, or a pharmaceutically acceptable salt, tautomer, stereoisomer, deuterated analog, or solvate of each thereof.

In some embodiments, the compound is an antipsychotic. Antipsychotics (sometimes referred to as neuroleptics) are a class of compounds primarily used to manage psychosis. In some embodiments, the antipsychotic is a 5-HT_{2A} antagonist. In some embodiments, the 5-HT_{2A} antagonist is lumateperone or olanzapine, or a pharmaceutically acceptable salt, tautomer, stereoisomer, deuterated analog, or solvate of each thereof.

Dopamine receptors (DR) are G protein-coupled receptors. DRs can be classified into D₁-like class when they couple to Gα_{s/olf} and stimulate adenylate cyclase production, or into D₂-like class when they couple to Gα_{i/o} and thus inhibit adenylate cyclase production. D₁-like receptors include D1 and D5 receptors; while D₂-like receptors include D₂, D₃, and D₄ receptors.

In some embodiments, the antipsychotic is a dopamine receptor (DR) antagonist. A DR antagonist is also referred to as anti-dopaminergic or dopamine antagonist. DR antagonist blocks DR by receptor antagonism. In some embodiments, the DR antagonist is butaclamol or a pharmaceutically acceptable salt, tautomer, stereoisomer, deuterated analog, or solvate thereof

In some embodiments, the compound is a dopamine receptor agonist. A DR agonist is also referred to as dopaminergic, or dopamine agonist. A DR agonist activates all or some of the dopamine receptors. A DR agonist that binds to and activates D 1 receptor is referred to as D1 receptor agonist; and a DR agonist that binds to and activates D5 receptor is referred to as D5 receptor agonist. Where a drug binds to and activates DR but only with partial efficacy as compared to “pure” agonist, the drug is referred to as a “DR partial agonist.” In some embodiments, the compound is dopaminergic.

In some embodiments, the dopamine receptor agonist is apomorphine or a pharmaceutically acceptable salt, tautomer, stereoisomer, deuterated analog, or solvate thereof. In some embodiments, the DR agonist is SKF-81297 or a pharmaceutically acceptable salt, tautomer, stereoisomer, deuterated analog, or solvate thereof. In some embodiments, the dopamine agonist is rotigotine or a pharmaceutically acceptable salt, tautomer, stereoisomer, deuterated analog, or solvate thereof

In some embodiments, the compound is DiDspartial agonist, D₁ receptor agonist, or D₃ agonist. In some embodiments, the dopaminergic is a DiDspartial agonist. D₁D₅ partial agonist refers to an agent that binds to and activate both D₁ and D₅ receptors but not as strongly as the “pure” D₁ or D₅ agonists. In some embodiments, the D₁D₅ partial agonist is SKF-38,393 or a pharmaceutically acceptable salt, tautomer, stereoisomer, deuterated analog, or solvate thereof.

In some embodiments, the dopaminergic is a D₁ receptor agonist. In some embodiments, the D₁ receptor agonist is A77636 or a pharmaceutically acceptable salt, tautomer, stereoisomer, deuterated analog, or solvate thereof.

In some embodiments, the dopaminergic is a D₃ agonist. In some embodiments, the D₃ agonist is 7-hydroxy-PIPAT (7-hydroxy-24N-propyl-N-(3′-iodo-2′-propenypaminoltetralin) or a pharmaceutically acceptable salt, tautomer, stereoisomer, deuterated analog, or solvate thereof.

In some embodiments, the antipsychotic is a 5-HT_{2A} antagonist and dopamine receptor agonist. In some embodiments, the compound is a 5-HT_{2A} antagonist and dopamine receptor agonist. In some embodiments, the 5-HT_{2A} antagonist and dopamine receptor agonist is an agent that modulates both the 5-HT_{2A} receptor and dopamine receptor. In some embodiments, the 5-HT_{2A} antagonist and dopamine receptor agonist is desmethylclozapine or a pharmaceutically acceptable salt, tautomer, stereoisomer, deuterated analog, or solvate thereof

In some embodiments, the compound is a serotonergic. Serotonergic refers to an agent that modulates or affects serotonin. Serotonin is a neurotransmitter that activates the serotonin receptors, a group of G protein-coupled receptor and ligand-gated ion channels found in the central and peripheral nervous system. Serotonin receptors are also referred to as 5-HT receptors. Serotonin receptors are classified into 5-HT_{1A}, 5-HT_{2B}, 5-HT₆, among many other subtypes. In some embodiments, the serotonergic is a 5-HT_{1A} agonist. In some embodiments, the 5-HT_{1A} agonist is S-14,506 or a pharmaceutically acceptable salt, tautomer, stereoisomer, deuterated analog, or solvate thereof. In some embodiments, the 5-HT_{1A} agonist is 8-hydroxy-PIPAT (8-hydroxy-2-[N-propyl-N-(3′-iodo-2′-propenypamino]tetralin), or a pharmaceutically acceptable salt, tautomer, stereoisomer, deuterated analog, or solvate thereof. In some embodiments, the serotonergic is a 5-HT₆ partial agonist. In some embodiments, the serotonergic is a 5-HT_{2B} antagonist. In some embodiments, the 5-HT_{2b} antagonist is LY-266,097, or a pharmaceutically acceptable salt, tautomer, stereoisomer, deuterated analog, or solvate thereof. In some embodiments, the 5-HT₆ partial agonist is EMD-386088 or a pharmaceutically acceptable salt, tautomer, stereoisomer, deuterated analog, or solvate thereof. In some embodiments, the compound is 5-HT_{1A} agonist, 5-HT₆ partial agonist, or 5-HT_{2b} antagonist.

In some embodiments, the compound is an iron chelator. In some embodiments, the compound is phenothiazine, EGFR inhibitor, or kinase inhibitor.

In some embodiments, the iron chelator is a kinase inhibitor. Protein kinases are ubiquitous cell surface and intracellular proteins that serve cell signaling functions involved in metabolism, injury responses, adaption, growth, and differentiation. A kinase inhibitor binds to and/or inhibits and/or modulates the activity of a kinases or kinases.

In some embodiments, the compound is Tpl2/MAP3K8 inhibitor, PKCβII inhibitor, PKC inhibitor, mixed kinase inhibitor, ALK inhibitor, FAK inhibitor, Ack1/ALK inhibitor, GSK-3 inhibitor, MPS1 kinase inhibitor, Mps1 inhibitor, Tyrosine kinase inhibitor, p38 inhibitor, GSK-3/CDK inhibitor, PIP5K1C inhibitor, angiokinase inhibitor, RTK inhibitor, HER1/2 kinase inhibitor, or PNKP inhibitor.

In some embodiments, the kinase inhibitor is a Tpl2/MAP3K8 inhibitor. Tumor Progression Locus 2 (Tpl2) and mitogen-activated protein kinase kinase kinase 8 (MAP3K8) are related serine/threonine kinases implicated in inflammation and pathological vascular angiogenesis. Tpl2/MAP3K8 inhibitor is an agent that modulates these enzymes and/or can inhibit their activities. In some embodiments, the Tpl2/MAP3K8 inhibitor is Cot inhibitor-2, or a pharmaceutically acceptable salt, tautomer, stereoisomer, deuterated analog, or solvate thereof.

In some embodiments, the kinase inhibitor is a PKC inhibitor. Protein kinase C (PKC) is a prototypical class of serine/threonine kinases that are specific signaling molecules linking multiple cellular processes to, for example, cancer. A PKC inhibitor is an agent that modulates PKC, thereby inhibiting their activities. In some embodiments, the PKC inhibitor is Go 6983 or a pharmaceutically acceptable salt, tautomer, stereoisomer, deuterated analog, or solvate thereof.

In some embodiments, the kinase inhibitor is a PKCβII inhibitor. PKCβII is a subclass and a splice variant of the “classic” PKC kinase. Elevated PKCβII levels relative to normal colonic tissue have been reported both during the initial stages of tumorigenesis and in colonic carcinomas. A PKCβII inhibitor is an agent that modulates PKCβII and can inhibit its activity. In some embodiments, the PKCβII inhibitor is CGP 53353 or a pharmaceutically acceptable salt, tautomer, stereoisomer, deuterated analog, or solvate thereof.

In some embodiments, the kinase inhibitor is a mixed lineage kinase (MLK) inhibitor. The MLK is a class of serine/threonine kinases that act as mitogen activated protein kinase kinase kinases (MAP3Ks). An MLK inhibitor is an agent that modulates MLK and can inhibit its activities. In some embodiments, the mixed lineage kinase inhibitor is URMC-099 or a pharmaceutically acceptable salt, tautomer, stereoisomer, deuterated analog, or solvate thereof.

In some embodiments, the kinase inhibitor is an anaplastic lymphoma kinase (ALK) inhibitor. ALK, also referred to as ALK tyrosine kinase receptor or CD246 (cluster of differentiation 246), is an enzyme that plays a vital role in the normal development and function of the nervous system. An ALK inhibitor modulates ALK and can thus inhibit its activities. In some embodiments, the ALK inhibitor is NVP-TAE684 or a pharmaceutically acceptable salt, tautomer, stereoisomer, deuterated analog, or solvate thereof.

In some embodiments, the kinase inhibitor is a focal adhesion kinase (FAK) inhibitor. FAK is a non-receptor tyrosine kinase that mediates signaling downstream of inte grin engagement of the extracellular matrix, and regulates cell survival, proliferation, and migration. A FAK inhibitor modulates FAK, thereby inhibiting its activities. In some embodiments, the FAK inhibitor is PND-1186 or a pharmaceutically acceptable salt, tautomer, stereoisomer, deuterated analog, or solvate thereof.

In some embodiments, the kinase inhibitor is an Ack1/ALK inhibitor. Ack1 is a type of tyrosine kinase implicated in a wide variety of human cancers. An Ack1/ALK inhibitor modulates and/or binds to Ack1 as well as to ALK, thereby inhibiting their activities. In some embodiments, the Ack1/ALK inhibitor is KRCA 0008 or a pharmaceutically acceptable salt, tautomer, stereoisomer, deuterated analog, or solvate thereof.

In some embodiments, the kinase inhibitor is a glycogen synthase kinase-3 (GSK-3) inhibitor. GSK-3 is a serine/threonine kinase whose aberrant activities have been linked with diseases such as diabetes, inflammation, and neurodegenerative, and psychiatric disorders. A GSK-3 inhibitor modulates GSK-3, thereby inhibiting its activities. In some embodiments, the GSK-3 inhibitor is GSK-3-inhibitor-IX or a pharmaceutically acceptable salt, tautomer, stereoisomer, deuterated analog, or solvate thereof.

In some embodiments, the kinase inhibitor is a monopolar spindle 1 (Mps1) kinase inhibitor. Mps1 is a regulator of the spindle assembly checkpoint (SAC) and has been associated with several types of tumors. Mps1 kinase inhibitor modulates the Mpsl kinase and inhibits its activities. In some embodiments, the Mps1 kinase inhibitor is Mps1-IN-1 or a pharmaceutically acceptable salt, tautomer, stereoisomer, deuterated analog, or solvate thereof. In some embodiments, the Mps1 inhibitor is MPI-0479605 or a pharmaceutically acceptable salt, tautomer, stereoisomer, deuterated analog, or solvate thereof.

In some embodiments, the kinase inhibitor is a tyrosine kinase inhibitor. Tyrosine kinases are enzymes associated with the activation of proteins by signal transduction cascades. A tyrosine kinase inhibitor modulates a tyrosine kinase and inhibits its activities. In some embodiments, the tyrosine kinase inhibitor is Masitinib (AB1010) or a pharmaceutically acceptable salt, tautomer, stereoisomer, deuterated analog, or solvate thereof

In some embodiments, the kinase inhibitor is a p38 inhibitor. The mitogen-activated protein kinase p38 is implicated in the regulation of inflammatory cytokine production. A p38 inhibitor modulates p38 kinase and inhibits its activities. In some embodiments, the p38 inhibitor is EO-1428 or a pharmaceutically acceptable salt, tautomer, stereoisomer, deuterated analog, or solvate thereof

In some embodiments, the kinase inhibitor is a GSK-3/CDK inhibitor. Cyclin-dependent kinase (CDK) are protein kinases characterized by needing a “cyclin” that provides domains essential for enzymatic activity. CDK is important in the control of cell divisions. A GSK-3/CDK inhibitor modulates GSK-3 and CDK, thereby inhibiting their activities. In some embodiments, the GSK-3/CDK inhibitor is indirubin-3-monoxime or a pharmaceutically acceptable salt, tautomer, stereoisomer, deuterated analog, or solvate thereof

In some embodiments, the kinase inhibitor is a phosphatidylinositol-4-phosphate 5-kinase Type 1 gamma (PIP5K1C) inhibitor. PIP5K1C is a lipid kinase implicated in the regulation of receptor-mediated calcium signaling in multiple tissues. A PIP5K1C inhibitor modulates PIP5K1C and inhibits its activities. In some embodiments, the PIP5K1C inhibitor is UNC-3230 or a pharmaceutically acceptable salt, tautomer, stereoisomer, deuterated analog, or solvate thereof.

In some embodiments, the kinase inhibitor is an angiokinase inhibitor. Angiokinase inhibitors are compounds that can inhibit the VEGF pathway as well as target other important signaling pathways of angiogenesis. In some embodiments, the angiokinase inhibitor is motesanib or a pharmaceutically acceptable salt, tautomer, stereoisomer, deuterated analog, or solvate thereof.

In some embodiments, the kinase inhibitor is a receptor tyrosine kinase (RTK) inhibitor. RTKs are essential in the regulation of cellular processes such as proliferation, differentiation, cell survival, apoptosis, and metabolism. RTK inhibitors modulate RTKs and inhibit their activities. In some embodiments, the RTK inhibitor is linifanib, or a pharmaceutically acceptable salt, tautomer, stereoisomer, deuterated analog, or solvate thereof

In some embodiments, the kinase inhibitor is a HER1/2 kinase inhibitor. HER1 and HER2 are proteins found on certain types of cells that bind to human epidermal growth factor. The HER1 protein is involved in cell signaling that is critical for cell division and survival; HER2 is also implicated in tumor growth. A HER1/2 kinase inhibitor modulates HER1 and HER2 kinases and inhibit their activities. In some embodiments, the HER1/2 kinase inhibitor is BMS-599626 or a pharmaceutically acceptable salt, tautomer, stereoisomer, deuterated analog, or solvate thereof.

In some embodiments, the kinase inhibitor is a polynucleotide kinase/phosphatase (PNKP) inhibitor. PNKP is an enzyme responsible for the repairing of DNA and rejoining of DNA single- and double-strand breaks. A PNKP inhibitor modulates PNKP and inhibits its activities. In some embodiments, the PNKP inhibitor is A12B4C3 or a pharmaceutically acceptable salt, tautomer, stereoisomer, deuterated analog, or solvate thereof.

In some embodiments, the compound is antiarrhythmic, antispasmodic, or antipruritc.

In some embodiments, the iron chelator is a phenothiazine. In some embodiments, the phenothiazine is an antiarrhythmic. Arai iarrnytil tnie refers to a compound that is capable of preve3 ting or treating abnormal heartbeats. In some embodiments, the antiarrhythmic is ethacizine or a pharmaceutically acceptable salt, tautomer, stereoisomer, deuterated analog, or solvate thereof

In some embodiments, the phenothiazine is an antispasmodic. Antispasmodic is a class of compounds that suppresses muscle spasms. In some embodiments, the antispasmodic is fenoverine or a pharmaceutically acceptable salt, tautomer, stereoisomer, deuterated analog, or solvate thereof

In some embodiments, the phenothiazine is an antipruritic. Antipruritic refers to a class of medication that inhibits itching due to, for example, sunburns, allergic reactions, eczema, psoriasis, chickenpox, fungal infections, etc. In some embodiments, the antipruritc is trimeprazine or a pharmaceutically acceptable salt, tautomer, stereoisomer, deuterated analog, or solvate thereof

In some embodiments, the iron chelator is cyamemazine, perphenazine, piperacetazine, mepazine, or levomepromazine, or a pharmaceutically acceptable salt, tautomer, stereoisomer, deuterated analog, or solvate of each thereof.

In some embodiments, the iron chelator is an epidermal growth factor receptor (EGFR) inhibitor. In some embodiments, the compound is an EGFR inhibitor. An EGFR inhibitor is a compound that can bind to EFGR and slow down or stop cell growth. In some embodiments, the EGFR inhibitor is CGP 52411, osimertinib, CNX-2006, wz4002, olmutinib, or lapatinib, or a pharmaceutically acceptable salt, tautomer, stereoisomer, deuterated analog, or solvate of each thereof.

In some embodiments, the iron chelator is an EGFR and ErbB2 inhibitor. In some embodiments, the compound is an EGFR and ErbB2 inhibitor. ErbB2 is also referred to as HER2. An EGFR and ErbB2 inhibitor is an agent that binds to EGFR and ErbB2 and inhibits their activities. In some embodiments, the EGFR and ErbB2 inhibitor is lapatinib or a pharmaceutically acceptable salt, tautomer, stereoisomer, deuterated analog, or solvate thereof

In some embodiments, the compound is an opioid receptor antagonist or δ receptor antagonist. In some embodiments, the compound is an opioid receptor antagonist. Opioid receptors are a group of inhibitory G protein-coupled receptors with opioids as ligands. An opioid receptor antagonist is an agent that inhibits the opioid receptor. In some embodiments, the opioid receptor antagonist is N-benzylnaltrindole or a pharmaceutically acceptable salt, tautomer, stereoisomer, deuterated analog, or solvate thereof. In some embodiments, the opioid receptor antagonist is N-benzylnaltrindole hydrochloride.

In some embodiments, the compound is a δ receptor antagonist. δ receptor is also referred to as the δ-opioid receptor, which is an inhibitory G-protein coupled receptor. A δ receptor antagonist is a compound that inhibits the δ receptor. In some embodiments, the δ receptor antagonist is naltriben, SDM25N, naltrindole, or a pharmaceutically acceptable salt, tautomer, stereoisomer, deuterated analog, or solvate of each thereof.

In some embodiments, the compound is a neuromodulator. In some embodiments, the compound is sigma receptor, NMDA antagonist, triple monoamine reuptake inhibitor, or NPY Y₅ antagonist.

In some embodiments, the neuromodulator acts on a sigma receptor (or σ receptor). Sigma receptors are a type of protein cell surface receptors that bind to certain ligands and have similar pharmacological behaviors. In some embodiments, the neuromodulator is a sigma receptor agonist. A sigma receptor agonist activates the sigma receptor when bound. In some embodiments, the sigma receptor is bromantane or a pharmaceutically acceptable salt, tautomer, stereoisomer, deuterated analog, or solvate thereof.

In some embodiments, the neuromodulator is an NMDA antagonist. N-methyl-D-aspartate (NMDA) receptors are glutamate-gated cation channels critical for the learning and memory development. NMDA antagonist is an agent that binds to the NMDA receptor and inhibits its activities. In some embodiments, the NMDA antagonist is Ro 04-5595 or a pharmaceutically acceptable salt, tautomer, stereoisomer, deuterated analog, or solvate thereof

In some embodiments, the neuromodulator is a triple monoamine reuptake inhibitor. Triple monoamine reuptake inhibitors simultaneously inhibit serotonin (5-HT), norepinephrine (NE) and dopamine reuptake, and are contemplated to be important for the pathophysiology of, for example, depression. In some embodiments, the triple monoamine reuptake inhibitor is diclofensine or a pharmaceutically acceptable salt, tautomer, stereoisomer, deuterated analog, or solvate thereof

In some embodiments, the neuromodulator is a neuropeptide Y (NPY) Y₅ antagonist. NPY is present in the hypothalamus and plays a critical role in the control of food intake. NPY Y₅ antagonist is a class of medication that inhibits the activities of NPY Y₅ subtype. In some embodiments, the NPY Y₅ antagonist is NPY-5RA972 or a pharmaceutically acceptable salt, tautomer, stereoisomer, deuterated analog, or solvate thereof.

In some embodiments, the compound is an S1P₃ receptor antagonist. Sphingosine 1-phosphate (S1P) receptors are G protein-coupled receptors important for the induction of biological responses in certain tissues. S1P₃ receptor antagonist is a compound that modulates and/or inhibits a specific SIP receptor, S1P₃. In some embodiments, the S1P₃ receptor antagonist is TY 52156 or a pharmaceutically acceptable salt, tautomer, stereoisomer, deuterated analog, or solvate thereof.

In some embodiments, the compound is an S1P₂ receptor antagonist. S1P₂ receptor antagonist is an agent that modulates and/or inhibits a specific S1P receptor (S1P₂). In some embodiments, the S1P₂ receptor antagonist is JTE-013 or a pharmaceutically acceptable salt, tautomer, stereoisomer, deuterated analog, or solvate thereof.

In some embodiments, the compound is a cell division cycle 25 (Cdc25) phosphatase inhibitor. Cdc25 proteins are dual-specific phosphatases regulating normal cell division and the cell response to DNA damage. Abnormal expression of Cdc25 has been detected in a number of tumors. Cdc25 phosphatase inhibitor is an agent that modulates Cdc25 and inhibits its activities. In some embodiments, the Cdc25 phosphatase inhibitor is NSC 663284 or a pharmaceutically acceptable salt, tautomer, stereoisomer, deuterated analog, or solvate thereof.

In some embodiments, the compound is an antimalarial. Antimalarial is an agent that can treat or prevent malaria infection. In some embodiments, the antimalarial is pyronaridine or a pharmaceutically acceptable salt, tautomer, stereoisomer, deuterated analog, or solvate thereof

In some embodiments, the compound is a PPARdelta antagonist. Peroxisome proliferator activated receptor delta (PPARδ or PPARdelta) is a type of protein whose overexpression is associated with several types of tumors. PPARdelta antagonist is an agent that inhibits the activities of PPARdelta. In some embodiments, the PPARdelta antagonist is GSK-0660 or a pharmaceutically acceptable salt, tautomer, stereoisomer, deuterated analog, or solvate thereof

In some embodiments, the compound is a nicotinamide phosphoribosyltransferase (NAMPT) activator. NAMPT is an enzyme responsible for catalysis of the nicotinamide adenine dinucleotide (NAD+) salvage pathway, which is pivotal in regulating energy metabolism. NAMPT activator activates and promotes the activities of NAMPT. In some embodiments, the NAMPT activator is P7C3 or a pharmaceutically acceptable salt, tautomer, stereoisomer, deuterated analog, or solvate thereof

In some embodiments, the compound is an autophagy activator. Autophagy is the process of removing unnecessary or dysfunctional components through a lysosome-dependent regulated mechanism. Autophagy activators are compounds that activate or stimulate the autography process. In some embodiments, the autophagy activator is STF-62247 or a pharmaceutically acceptable salt, tautomer, stereoisomer, deuterated analog, or solvate thereof.

In some embodiments, the compound is a neurogenesis activator. Neurogenesis is the process in which neurons are produced by neural stem cells. Neurogenesis activators are compounds that activate or stimulate the neurogenesis process. In some embodiments, the neurogenesis activator is neurodazine or a pharmaceutically acceptable salt, tautomer, stereoisomer, deuterated analog, or solvate thereof.

In some embodiments, the compound is a smoothened agonist. Smoothened is a protein key to the hedgehog signaling pathway, which finds criticality in brain development. Smoothened agonist is an agent that activates or stimulates the protein, smoothened, and aids the proliferation of survival and developing neurons. In some embodiments, the smoothened agonist is purmorphamine or a pharmaceutically acceptable salt, tautomer, stereoisomer, deuterated analog, or solvate thereof

In some embodiments, the compound is an STAT3 inhibitor. Signal Transducer and Activator of Transcription 3 (STAT3) is a transcription factor that regulates, for example, proliferation, metastasis, angiogenesis, immune response, chemoresistance, among other biological processes. STAT3 inhibitor modulates STAT3 and inhibits its activities. In some embodiments, the STAT3 inhibitor is ochromycinone or a pharmaceutically acceptable salt, tautomer, stereoisomer, deuterated analog, or solvate thereof.

In some embodiments, the compound is a lactate dehydrogenase A (LDHA) inhibitor. LDHA is a monomer of lactate dehydrogenase and is responsible for the catalysis of interconversion of pyruvate and L-lactate. LDHA inhibitor is an agent that modulates LDHA and inhibits its activities. In some embodiments, the LDHA inhibitor is NHI-2 or a pharmaceutically acceptable salt, tautomer, stereoisomer, deuterated analog, or solvate thereof

In some embodiments, the compound is an antiviral. An antiviral is a compound that can be useful for treating viral infections. In some embodiments, the antiviral is bonaphthone or TMC353121 or a pharmaceutically acceptable salt, tautomer, stereoisomer, deuterated analog, or solvate of each thereof

In some embodiments, the compound is a PDGFRbeta receptor inhibitor. Platelet-Derived Growth Factor Receptor beta (PDGFRβ or PDGFRbeta) is a receptor tyrosine kinase implicated in atherogenesis. A PDGFRbeta receptor inhibitor modulates the PDGFRbeta receptor and inhibits its activities. In some embodiments, the PDGFRbeta receptor inhibitor is su-16f or a pharmaceutically acceptable salt, tautomer, stereoisomer, deuterated analog, or solvate thereof

In some embodiments, the compound is an N-acetyltransferase 10 (NAT10) inhibitor. NAT10 is an enzyme regulator of mRNA acetylation and is found to be activated in many types of cancers. NAT10 inhibitor is an agent that modulates and inhibits NAT10 activities. In some embodiments, the NAT10 inhibitor is remodelin or a pharmaceutically acceptable salt, tautomer, stereoisomer, deuterated analog, or solvate thereof.

In some embodiments, the compound is a farnesoid X receptor (FXR) agonist. FXR is a metabolic nuclear receptor regulating cholesterol homeostasis, lipid metabolism, glucose metabolism, and intestinal microorganism. FXR agonist is an agent that activates or stimulates FXR activities. In some embodiments, the FXR agonist is fexaramine or a pharmaceutically acceptable salt, tautomer, stereoisomer, deuterated analog, or solvate thereof.

In some embodiments, the compound is a cystic fibrosis transmembrane regulator (CFTR) potentiator. CFTR is a protein that functions as a channel across the membrane of cells that produce mucus, sweat, saliva, tears, and digestive enzymes. CFTR potenitator functions by keeping open a gate within the protein in a correct conformation for sufficiently long time to allow for transport of chloride ions across the channel. In some embodiments, the CFTR potentiator is ivacaftor or a pharmaceutically acceptable salt, tautomer, stereoisomer, deuterated analog, or solvate thereof.

In some embodiments, the compound is a G protein-coupled receptor 139 (GPR139) agonist. GPR139 is an orphan G-protein-coupled receptor expressed in the central nervous system. GPR139 agonist is an agent activates the activities of GPR139. In some embodiments, the GPR139 agonist is TC-O-9311 or a pharmaceutically acceptable salt, tautomer, stereoisomer, deuterated analog, or solvate thereof.

In some embodiments, the compound is a histone deacetylase (HDAC) class II inhibitor. HDAC class II is a subset of HDAC which regulates differentiation processes, such as muscle and neuronal differentiation. HDAC class II inhibitors binds to HDAC class II and inhibit its activities. In some embodiments, the HDAC class II inhibitor is MC1568 or a pharmaceutically acceptable salt, tautomer, stereoisomer, deuterated analog, or solvate thereof.

In some embodiments, the compound is a multidrug resistance pump inhibitor. Multidrug resistance pump confers resistance to antibiotics. A multidrug resistance pump inhibitor modulates the multidrug resistance pump and inhibits its activities. In some embodiments, the multidrug resistance pump inhibitor is Ko 143 or a pharmaceutically acceptable salt, tautomer, stereoisomer, deuterated analog, or solvate thereof.

In some embodiments, the compound is a gamma secretase inhibitor. Gamma secretase is a multi-subunit protease that regulates intramembrane-cleaving. Gamma secretase inhibitor is an agent that modulates gamma secretase and inhibits its activities. In some embodiments, the gamma secretase inhibitor is nirogacestat or a pharmaceutically acceptable salt, tautomer, stereoisomer, deuterated analog, or solvate thereof.

In some embodiments, the compound is a dihydropyridine calcium channel blocker or a pharmaceutically acceptable salt, tautomer, stereoisomer, deuterated analog, or solvate thereof. In some embodiments, the dihydropyridine calcium channel blocker is azelnidipine or a pharmaceutically acceptable salt, tautomer, stereoisomer, deuterated analog, or solvate thereof. In some embodiments, the compound is azelnidipine or a pharmaceutically acceptable salt thereof. In some embodiments, the compound is azelnidipine.

In some embodiments, the compound is an estrogen receptor modulator.

In some embodiments, the compound is bazedoxifene or a pharmaceutically acceptable salt, tautomer, stereoisomer, deuterated analog, or solvate thereof. In some embodiments, the compound is bazedoxifene or a pharmaceutically acceptable thereof. In some embodiments, the compound is bazedoxifene.

In some embodiments, the compound is selected from those listed in Table 1A, or a pharmaceutically acceptable salt, tautomer, stereoisomer, deuterated analog, or solvate thereof.

TABLE 1A

| Ex. | | Compound |
|---|---|---|
| No. | Compound Structure | Identification |
| 1 | 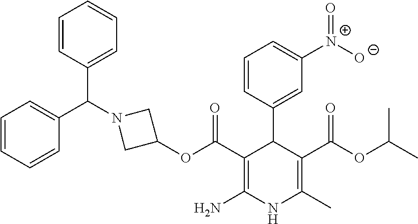 [SMILES: CC1=C(C(=O)OC(C)C)C(c2cccc([N+](=O)[O-])c2)C(C(=O)OC2CN(C(c3ccccc3)c3ccccc3)C2)=C(N)N1] | azelnidipine CalBlock |
| 2 | 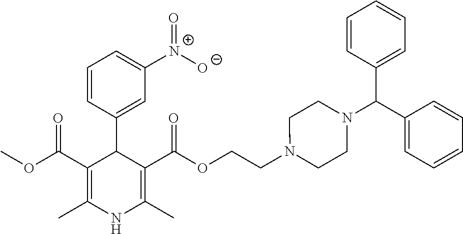 [SMILES: COC(=O)C1=C(C)NC(C)=C(C(=O)OCCN2CCN(C(c3ccccc3)c3ccccc3)CC2)C1c1cccc([N+](=O)[O-])c1] | manidipine Manyper |
| 3 | 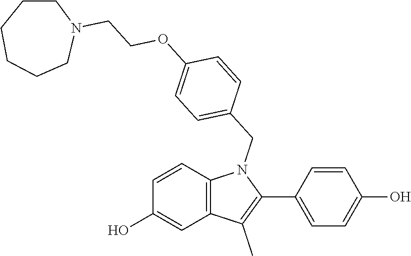 [SMILES: Cc1c(-c2ccc(O)cc2)n(Cc2ccc(OCCN3CCCCCC3)cc2)c2ccc(O)cc12] | bazedoxifene Viviant Conbriza |
| 5 | 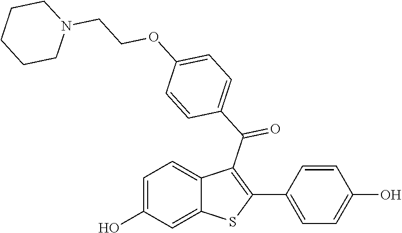 [SMILES: O=C(c1ccc(OCCN2CCCCC2)cc1)c1c(-c2ccc(O)cc2)sc2cc(O)ccc12] | raloxifene Evista |
| 10 | 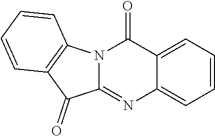 [SMILES: O=C1c2ccccc2-n2c1nc1ccccc1c2=O] | trypthanthrin (GRAS) |
| 11 | 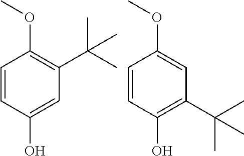 | butylated hydroxyanisole (GRAS) |
| 12 | 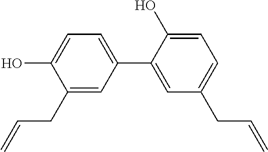 [SMILES: C=CCc1ccc(O)c(-c2ccc(O)c(CC=C)c2)c1] | honokiol (GRAS) |
| 14 | 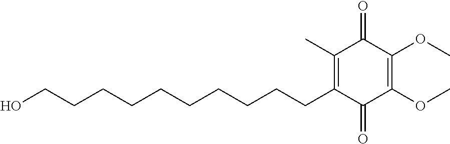 [SMILES: COC1=C(OC)C(=O)C(CCCCCCCCCCO)=C(C)C1=O] | idebenone |
| 15 | 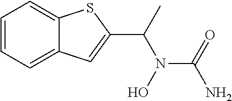 [SMILES: CC(c1cc2ccccc2s1)N(O)C(N)=O] | Zileuton Zylflo |
| 16 | 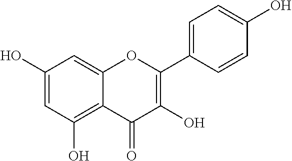 [SMILES: O=c1c(O)c(-c2ccc(O)cc2)oc2cc(O)cc(O)c12] | kaempferol (GRAS) |
| 18 | 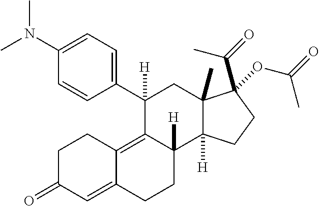 | ulipristal acetate/Ella |
| 19 | 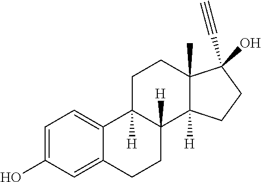 | ethinyl estradiol |
| 20 | 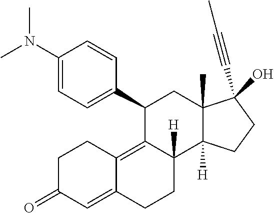 | Mifepristone Mifeprex |
| 21 | 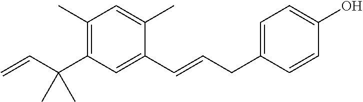 [SMILES: C=CC(C)(C)c1cc(/C=C/Cc2ccc(O)cc2)c(C)cc1C] | licochalcone (GRAS) |
| 22 | 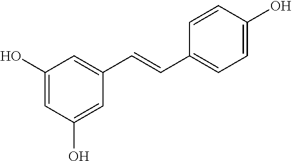 [SMILES: Oc1ccc(/C=C/c2cc(O)cc(O)c2)cc1] | resveratrol (GRAS) |
| 23 | 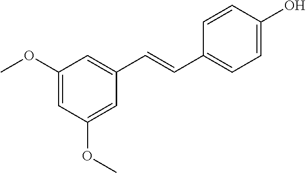 [SMILES: COc1cc(/C=C/c2ccc(O)cc2)cc(OC)c1] | pterostilbene (GRAS) |
| 24 | 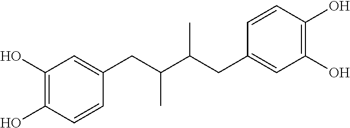 [SMILES: CC(Cc1ccc(O)c(O)c1)C(C)Cc1ccc(O)c(O)c1] | Nordihydro- guaiaretic acid (GRAS) |
| 25 | 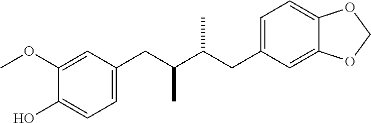 [SMILES: COc1cc(C[C@H](C)[C@H](C)Cc2ccc3c(c2)OCO3)ccc1O] | macelignan (GRAS) |
| 26 | 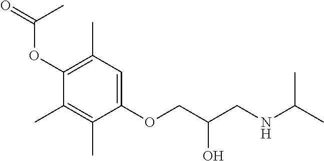 [SMILES: CC(=O)Oc1c(C)cc(OCC(O)CNC(C)C)c(C)c1C] | metipranolol |
| 28 | 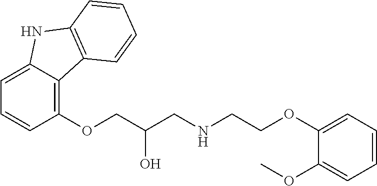 [SMILES: COc1ccccc1OCCNCC(O)COc1cccc2[nH]c3ccccc3c12] | carvedilol Coreg |
| 29 | 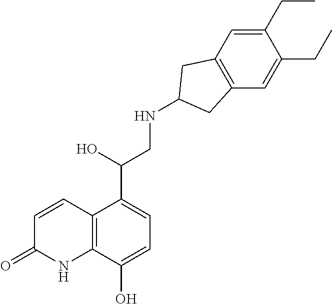 [SMILES: CCc1cc2c(cc1CC)CC(NCC(O)c1ccc(O)c3[nH]c(=O)ccc13)C2] | indacaterol Arcapta Neohaler |
| 30 | 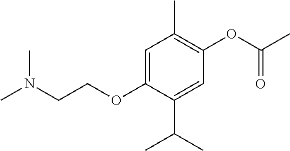 [SMILES: CC(=O)Oc1cc(C(C)C)c(OCCN(C)C)cc1C] | Moxisylyte Opilon |
| 31 | 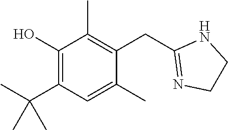 [SMILES: Cc1cc(C(C)(C)C)c(O)c(C)c1CC1=NCCN1] | oxymetazoline Afran |
| 32 | 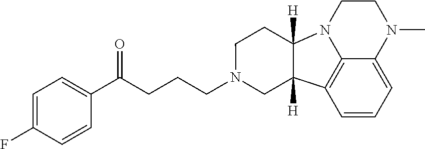 [SMILES: CN1CCN2c3c(cccc31)[C@@H]1CN(CCCC(=O)c3ccc(F)cc3)CC[C@@H]12] | lumateperone Caplyta |
| 33 | 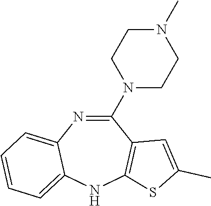 [SMILES: Cc1cc2c(s1)Nc1ccccc1N=C2N1CCN(C)CC1] | olanzapine Zyprexa |
| 34 | 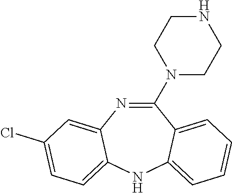 [SMILES: Clc1ccc2c(c1)N=C(N1CCNCC1)c1ccccc1N2] | desmethyl- clozapine Clozaril |
| 36 | 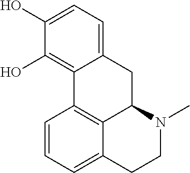 [SMILES: CN1CCc2cccc3c2[C@H]1Cc1ccc(O)c(O)c1-3] | apomorphine Apokyn |
| 41 | 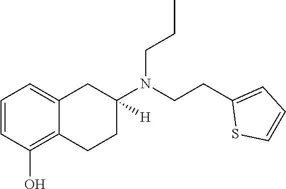 [SMILES: CCCN(CCc1cccs1)[C@H]1CCc2c(O)cccc2C1] | rotigotine Neupro |
| 42 | 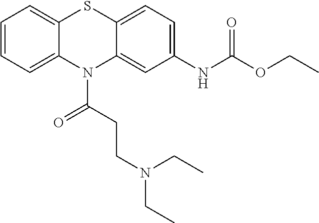 [SMILES: CCOC(=O)Nc1ccc2c(c1)N(C(=O)CCN(CC)CC)c1ccccc1S2] | ethacizine |
| 43 | 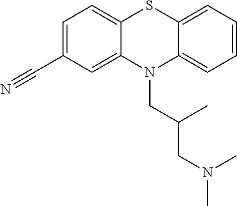 [SMILES: CC(CN(C)C)CN1c2ccccc2Sc2ccc(C#N)cc21] | cyamemazine |
| 44 | 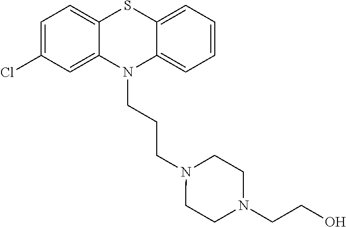 [SMILES: OCCN1CCN(CCCN2c3ccccc3Sc3ccc(Cl)cc32)CC1] | perphenazine |
| 45 | 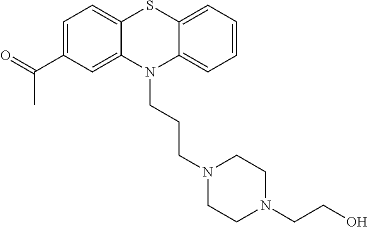 [SMILES: CC(=O)c1ccc2c(c1)N(CCCN1CCN(CCO)CC1)c1ccccc1S2] | piperacetazine |
| 46 | 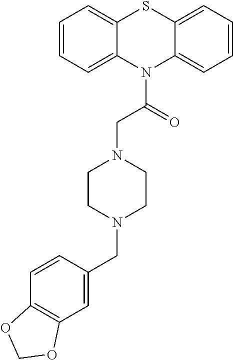 [SMILES: O=C(CN1CCN(Cc2ccc3c(c2)OCO3)CC1)N1c2ccccc2Sc2ccccc21] | fenoverine |
| 47 | 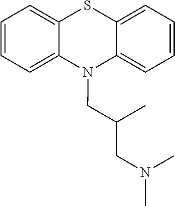 [SMILES: CC(CN(C)C)CN1c2ccccc2Sc2ccccc21] | trimeprazine |
| 48 | 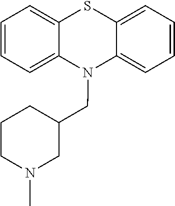 [SMILES: CN1CCCC(CN2c3ccccc3Sc3ccccc32)C1] | mepazine |
| 49 | 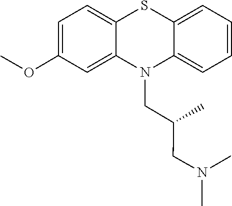 [SMILES: COc1ccc2c(c1)N(C[C@H](C)CN(C)C)c1ccccc1S2] | Levomepromazine |
| 60 | 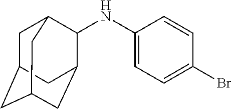 [SMILES: Brc1ccc(NC2C3CC4CC(C3)CC2C4)cc1] | bromantane Ladastan |
| 64 | 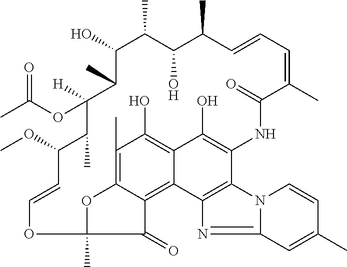 [SMILES: CO[C@H]1/C=C/O[C@@]2(C)Oc3c(C)c(O)c4c(O)c(c5c(nc6cc(C)ccn65)c4c3C2=O)NC(=O)/C(C)=C\C=C\[C@H](C)[C@H](O)[C@@H](C)[C@@H](O)[C@@H](C)[C@H](OC(C)=O)[C@@H]1C] | rifaximin |
| 65 | 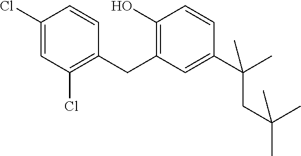 [SMILES: CC(C)(C)CC(C)(C)c1ccc(O)c(Cc2ccc(Cl)cc2Cl)c1] | clofoctol |
| 67 | 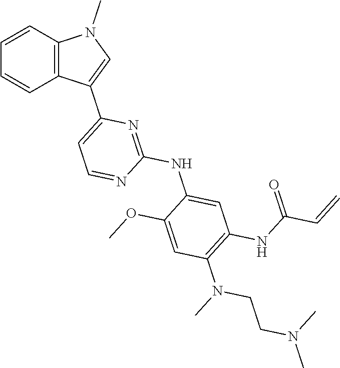 [SMILES: C=CC(=O)Nc1cc(Nc2nccc(-c3cn(C)c4ccccc34)n2)c(OC)cc1N(C)CCN(C)C] | osimertinib Tagrisso |
| 71 | 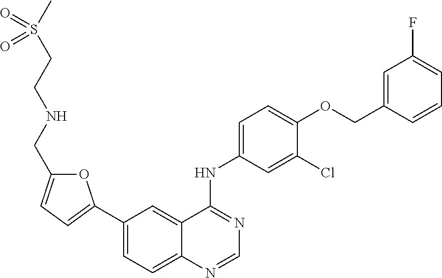 [SMILES: CS(=O)(=O)CCNCc1ccc(-c2ccc3ncnc(Nc4ccc(OCc5cccc(F)c5)c(Cl)c4)c3c2)o1] | lapatinib |
| 92 | 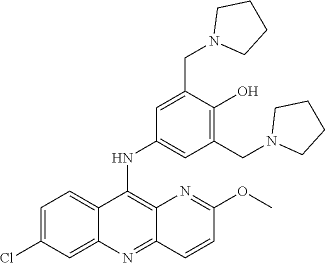 [SMILES: COc1ccc2nc3cc(Cl)ccc3c(Nc3cc(CN4CCCC4)c(O)c(CN4CCCC4)c3)c2n1] | pyronaridine |
| 105 | 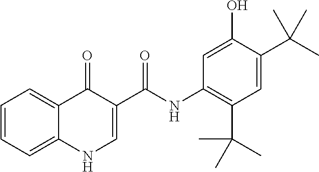 [SMILES: CC(C)(C)c1cc(C(C)(C)C)c(NC(=O)c2c[nH]c3ccccc3c2=O)cc1O] | ivacaftor |

In some embodiments, the compound is selected from those listed in Table 1B, or a pharmaceutically acceptable salt, tautomer, stereoisomer, deuterated analog, or solvate thereof

TABLE 1B

| Ex. | | Compound |
|---|---|---|
| No. | Compound Structure | Identification |
| 4 | 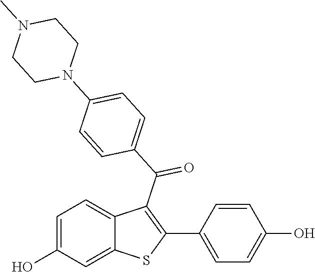 [SMILES: CN1CCN(c2ccc(C(=O)c3c(-c4ccc(O)cc4)sc4cc(O)ccc34)cc2)CC1] | Y-134 |
| 6 | 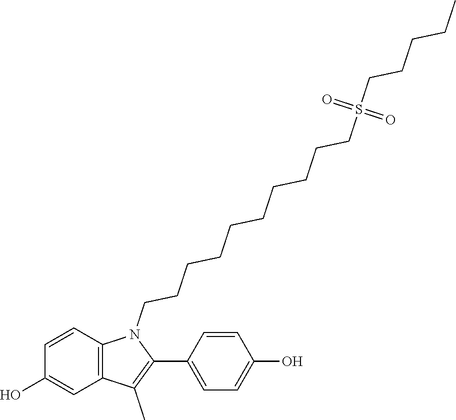 [SMILES: CCCCCS(=O)(=O)CCCCCCCCCCn1c(-c2ccc(O)cc2)c(C)c2cc(O)ccc21] | ZK-164015 |
| 7 | 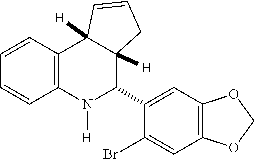 [SMILES: Brc1cc2c(cc1[C@@H]1Nc3ccccc3[C@@H]3C=CC[C@@H]31)OCO2] | G-15 |
| 8 | 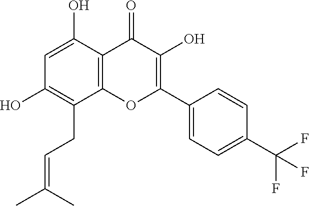 [SMILES: CC(C)=CCc1c(O)cc(O)c2c(=O)c(O)c(-c3ccc(C(F)(F)F)cc3)oc12] | SNG1153 |
| 9 | 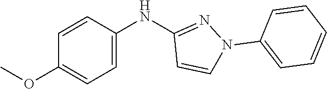 [SMILES: COc1ccc(Nc2ccn(-c3ccccc3)n2)cc1] | FPL 62064 |
| 13 | 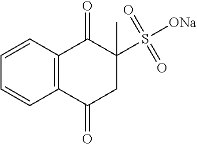 | menadione |
| 17 | 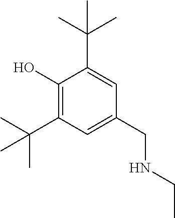 [SMILES: CCNCc1cc(C(C)(C)C)c(O)c(C(C)(C)C)c1] | LY 231617 |
| 27 | 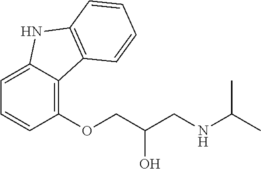 [SMILES: CC(C)NCC(O)COc1cccc2[nH]c3ccccc3c12] | carazolol |
| 35 | 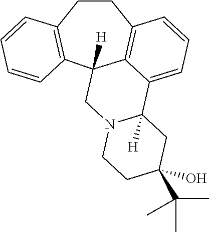 | butaclamol |
| 37 | 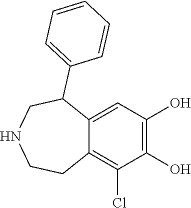 [SMILES: Oc1cc2c(c(Cl)c1O)CCNCC2c1ccccc1] | SKF-81297 |
| 38 | 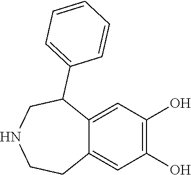 [SMILES: Oc1cc2c(cc1O)C(c1ccccc1)CNCC2] | SKF-38,393 |
| 39 | 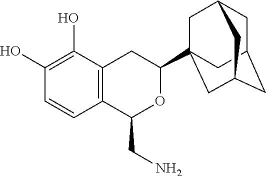 [SMILES: NC[C@@H]1O[C@H](C23CC4C[C@H](C2)C[C@H](C4)C3)Cc2c1ccc(O)c2O] | A77636 |
| 40 | 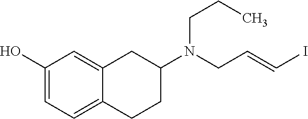 [SMILES: CCCN(C/C=C/I)C1CCc2ccc(O)cc2C1] | 7-hydroxy- PIPAT |
| 50 | 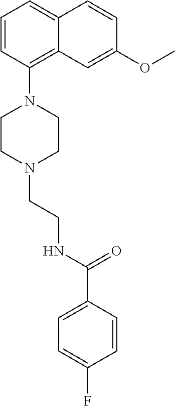 [SMILES: COc1ccc2cccc(N3CCN(CCNC(=O)c4ccc(F)cc4)CC3)c2c1] | S-14,506 |
| 51 | 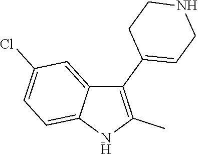 [SMILES: Cc1[nH]c2ccc(Cl)cc2c1C1=CCNCC1] | EMD-386088 |
| 52 | 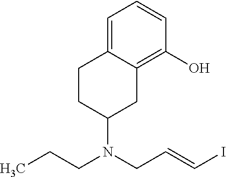 [SMILES: CCCN(C/C=C/I)C1CCc2cccc(O)c2C1] | 8-hydroxy- PIPAT |
| 53 | 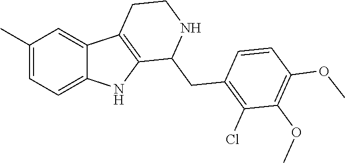 | LY-266,097 |
| 54 | 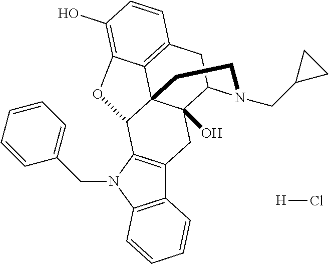 | N-Benzyl- naltrindole HCl |
| 55 | 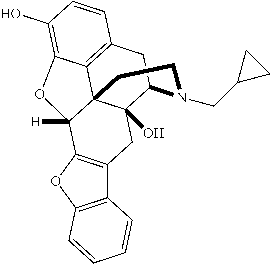 | Naltriben |
| 56 | 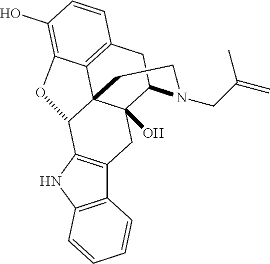 | SDM25N |
| 57 | 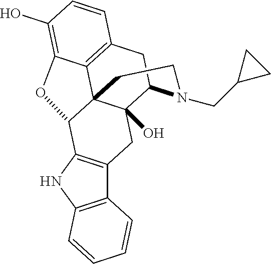 | Naltrindole |
| 58 | 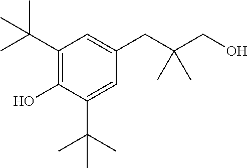 [SMILES: CC(C)(CO)Cc1cc(C(C)(C)C)c(O)c(C(C)(C)C)c1] | CGP-7930 |
| 59 | 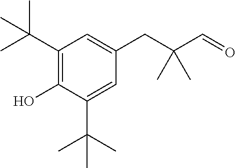 [SMILES: CC(C)(C=O)Cc1cc(C(C)(C)C)c(O)c(C(C)(C)C)c1] | CGP-13501 |
| 61 | 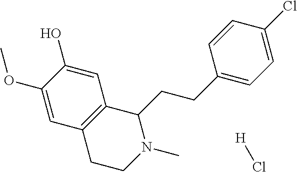 [SMILES: COc1cc2c(cc1O)C(CCc1ccc(Cl)cc1)N(C)CC2 ; Cl] | Ro 04-5595 |
| 62 | 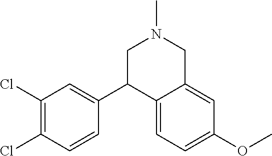 [SMILES: COc1ccc2c(c1)CN(C)CC2c1ccc(Cl)c(Cl)c1] | Diclofensine |
| 63 | 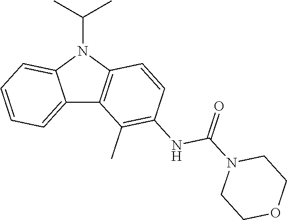 [SMILES: Cc1c(NC(=O)N2CCOCC2)ccc2c1c1ccccc1n2C(C)C] | NPY-5RA972 |
| 66 | 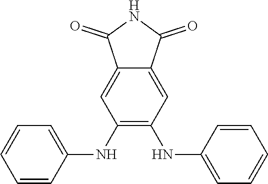 [SMILES: O=C1NC(=O)c2cc(Nc3ccccc3)c(Nc3ccccc3)cc21] | CGP 52411 |
| 68 | 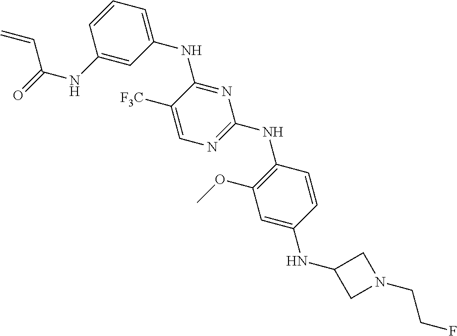 [SMILES: C=CC(=O)Nc1cccc(Nc2nc(Nc3ccc(NC4CN(CCF)C4)cc3OC)ncc2C(F)(F)F)c1] | CNX-2006 |
| 69 | 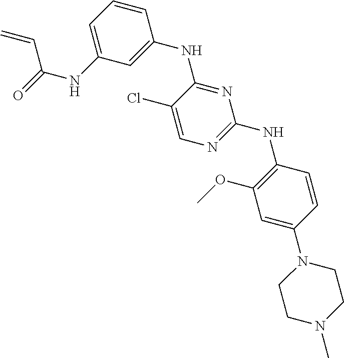 [SMILES: C=CC(=O)Nc1cccc(Nc2nc(Nc3ccc(N4CCN(C)CC4)cc3OC)ncc2Cl)c1] | wz4002 |
| 70 | 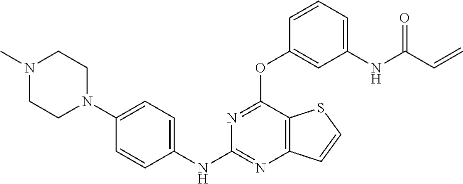 [SMILES: C=CC(=O)Nc1cccc(Oc2nc(Nc3ccc(N4CCN(C)CC4)cc3)nc3ccsc23)c1] | Olmutinib |
| 72 | 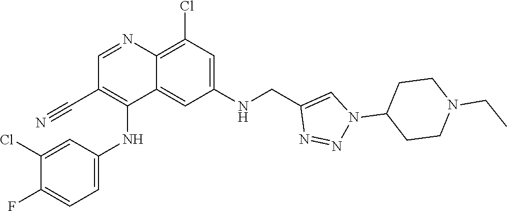 [SMILES: CCN1CCC(n2cc(CNc3cc(Cl)c4ncc(C#N)c(Nc5ccc(F)c(Cl)c5)c4c3)nn2)CC1] | Cot inhibitor-2 |
| 73 | 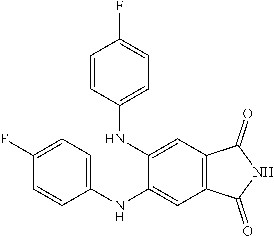 [SMILES: O=C1NC(=O)c2cc(Nc3ccc(F)cc3)c(Nc3ccc(F)cc3)cc21] | CGP 53353 |
| 74 | 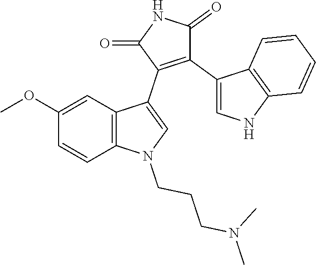 [SMILES: COc1ccc2c(c1)c(C1=C(c3c[nH]c4ccccc34)C(=O)NC1=O)cn2CCCN(C)C] | Go 6983 |
| 75 | 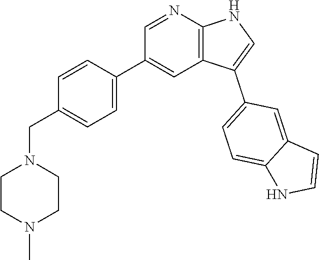 [SMILES: CN1CCN(Cc2ccc(-c3cnc4[nH]cc(-c5ccc6[nH]ccc6c5)c4c3)cc2)CC1] | URMC-099 |
| 76 | 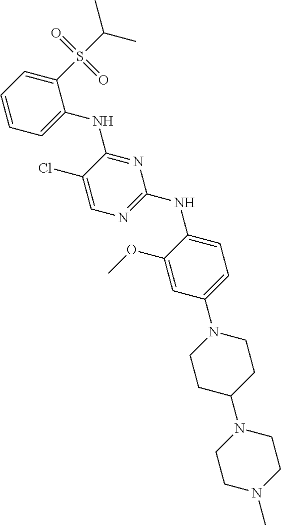 [SMILES: COc1cc(N2CCC(N3CCN(C)CC3)CC2)ccc1Nc1ncc(Cl)c(Nc2ccccc2S(=O)(=O)C(C)C)n1] | NVP-TAE684 |
| 77 | 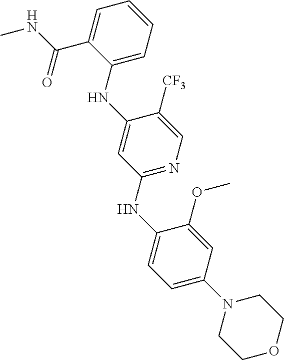 [SMILES: CNC(=O)c1ccccc1Nc1cc(Nc2ccc(N3CCOCC3)cc2OC)ncc1C(F)(F)F] | PND-1186 (VS-4718) |
| 78 | 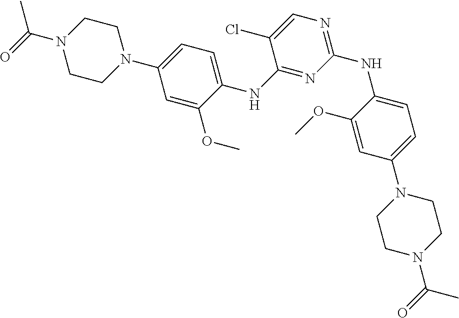 [SMILES: COc1cc(N2CCN(C(C)=O)CC2)ccc1Nc1ncc(Cl)c(Nc2ccc(N3CCN(C(C)=O)CC3)cc2OC)n1] | KRCA 0008 |
| 79 | 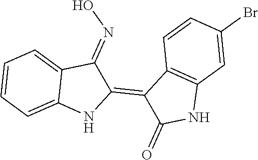 [SMILES: O=C1Nc2cc(Br)ccc2/C1=C1/Nc2ccccc2/C1=N\O] | GSK-3- inhibitor-IX |
| 80 | 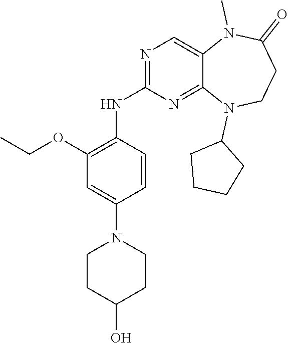 [SMILES: CCOc1cc(N2CCC(O)CC2)ccc1Nc1ncc2c(n1)N(C1CCCC1)CCC(=O)N2C] | Mps1-IN-1 |
| 81 | 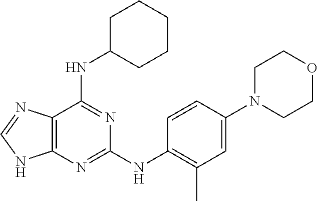 [SMILES: Cc1cc(N2CCOCC2)ccc1Nc1nc(NC2CCCCC2)c2nc[nH]c2n1] | MPI-0479605 |
| 82 | 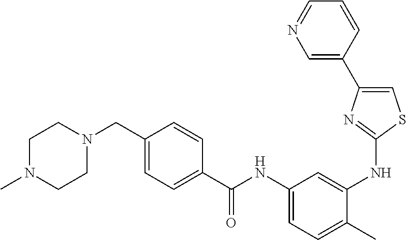 [SMILES: Cc1ccc(NC(=O)c2ccc(CN3CCN(C)CC3)cc2)cc1Nc1nc(-c2cccnc2)cs1] | Masitinib (AB1010) |
| 83 | 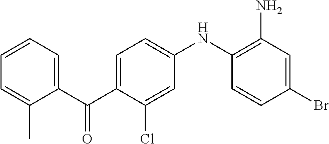 [SMILES: Cc1ccccc1C(=O)c1ccc(Nc2ccc(Br)cc2N)cc1Cl] | EO-1428 |
| 84 | 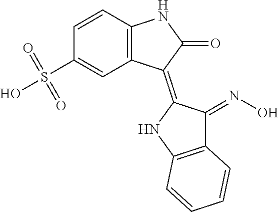 [SMILES: O=C1Nc2ccc(S(=O)(=O)O)cc2/C1=C1\Nc2ccccc2\C1=N/O] | indirubin-3- monoxime |
| 85 | 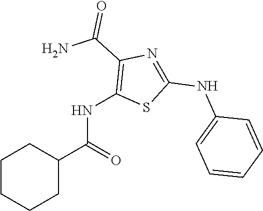 [SMILES: NC(=O)c1nc(Nc2ccccc2)sc1NC(=O)C1CCCCC1] | UNC-3230 |
| 86 | 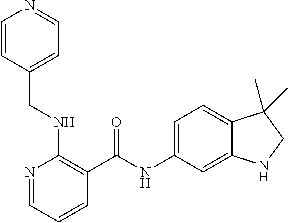 [SMILES: CC1(C)CNc2cc(NC(=O)c3cccnc3NCc3ccncc3)ccc21] | Motesanib |
| 87 | 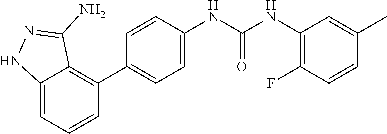 [SMILES: Cc1ccc(F)c(NC(=O)Nc2ccc(-c3cccc4[nH]nc(N)c34)cc2)c1] | Linifanib |
| 88 | 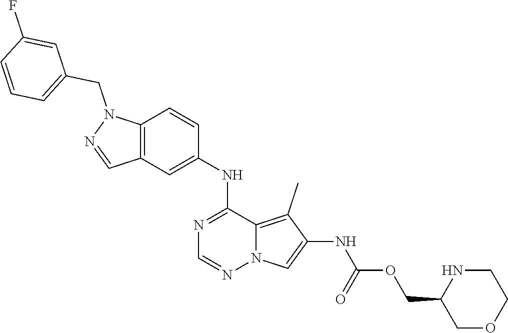 [SMILES: Cc1c(NC(=O)OC[C@@H]2COCCN2)cn2ncnc(Nc3ccc4c(cnn4Cc4cccc(F)c4)c3)c12] | BMS-599626 |
| 89 | 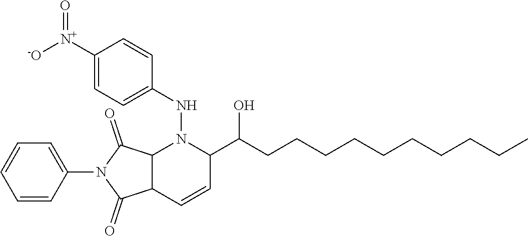 | A12B4C3 |
| 90 | 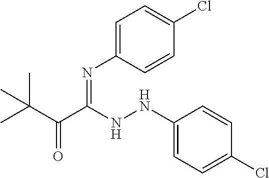 [SMILES: CC(C)(C)C(=O)/C(=N/c1ccc(Cl)cc1)NNc1ccc(Cl)cc1] | TY 52156 |
| 91 | 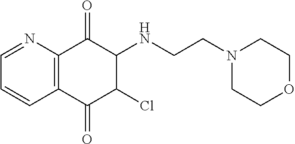 [SMILES: O=C1c2cccnc2C(=O)C(NCCN2CCOCC2)C1Cl] | NSC 663284 |
| 93 | 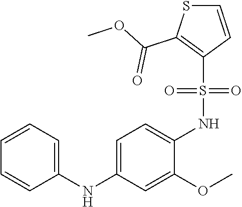 [SMILES: COC(=O)c1sccc1S(=O)(=O)Nc1ccc(Nc2ccccc2)cc1OC] | GSK-0660 |
| 94 | 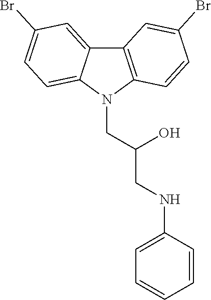 [SMILES: OC(CNc1ccccc1)Cn1c2ccc(Br)cc2c2cc(Br)ccc21] | P7C3 |
| 95 | 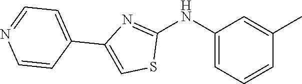 [SMILES: Cc1cccc(Nc2nc(-c3ccncc3)cs2)c1] | STF-62247 |
| 96 | 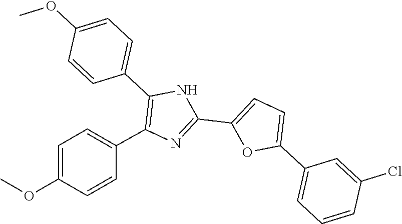 [SMILES: COc1ccc(-c2nc(-c3ccc(-c4cccc(Cl)c4)o3)[nH]c2-c2ccc(OC)cc2)cc1] | neurodazine |
| 97 | 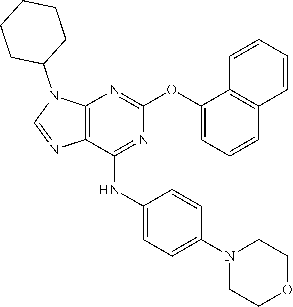 [SMILES: c1ccc2c(Oc3nc(Nc4ccc(N5CCOCC5)cc4)c4ncn(C5CCCCC5)c4n3)cccc2c1] | Purmorphamine |
| 98 | 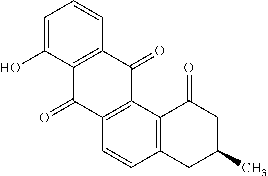 [SMILES: C[C@@H]1CC(=O)c2c(ccc3c2C(=O)c2cccc(O)c2C3=O)C1] | ochromycinone |
| 99 | 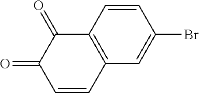 [SMILES: O=C1C=Cc2cc(Br)ccc2C1=O] | bonaphthone |
| 100 | 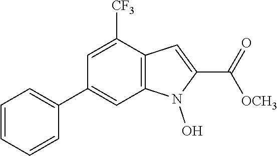 [SMILES: COC(=O)c1cc2c(C(F)(F)F)cc(-c3ccccc3)cc2n1O] | NHI-2 |
| 101 | 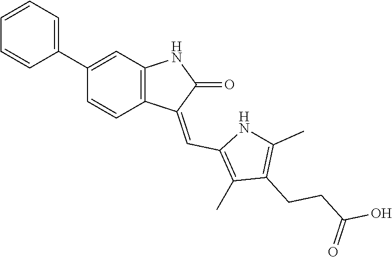 [SMILES: Cc1[nH]c(/C=C2\C(=O)Nc3cc(-c4ccccc4)ccc32)c(C)c1CCC(=O)O] | su-16f |
| 102 | 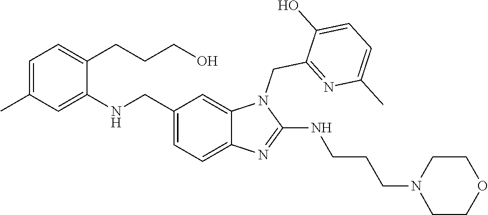 [SMILES: Cc1ccc(CCCO)c(NCc2ccc3nc(NCCCN4CCOCC4)n(Cc4nc(C)ccc4O)c3c2)c1] | TMC353121 |
| 103 | 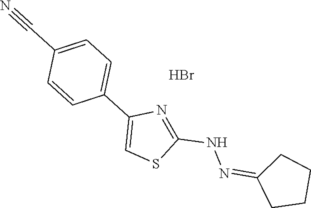 [SMILES: N#Cc1ccc(-c2csc(NN=C3CCCC3)n2)cc1 ; Br] | remodelin |
| 104 | 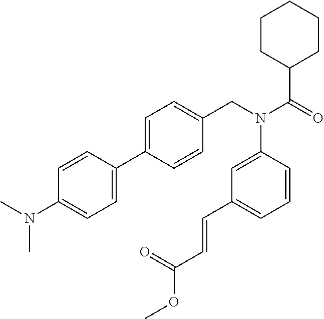 [SMILES: COC(=O)/C=C/c1cccc(N(Cc2ccc(-c3ccc(N(C)C)cc3)cc2)C(=O)C2CCCCC2)c1] | Fexaramine |
| 106 | 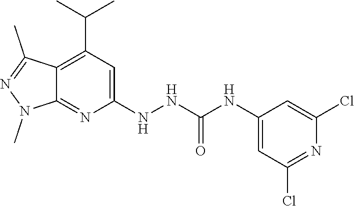 [SMILES: Cc1nn(C)c2nc(NNC(=O)Nc3cc(Cl)nc(Cl)c3)cc(C(C)C)c12] | JTE-013 |
| 107 | 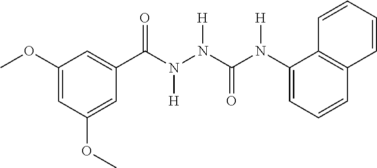 [SMILES: COc1cc(OC)cc(C(=O)NNC(=O)Nc2cccc3ccccc23)c1] | TC-O-9311 |
| 108 | 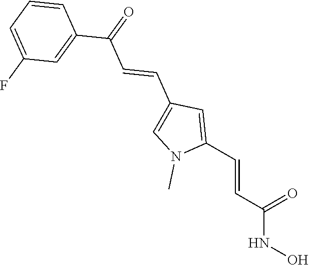 [SMILES: Cn1cc(/C=C/C(=O)c2cccc(F)c2)cc1/C=C/C(=O)NO] | MC1568 |
| 109 | 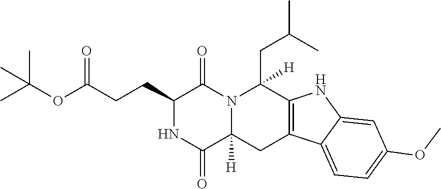 [SMILES: COc1ccc2c3c([nH]c2c1)[C@H](CC(C)C)N1C(=O)[C@H](CCC(=O)OC(C)(C)C)NC(=O)[C@@H]1C3] | Ko 143 |
| 110 | 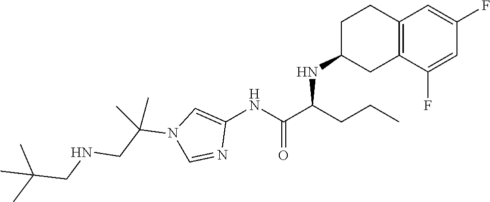 [SMILES: CCC[C@H](N[C@H]1CCc2cc(F)cc(F)c2C1)C(=O)Nc1cn(C(C)(C)CNCC(C)(C)C)cn1] | Nirogacestat |

Compounds of Tables 1A and 1B are commercially available, for example, from Sigma Aldrich or similar chemical providers, or can be made according to methods known in the art.

Also provided herein, in some embodiments, are pharmaceutical compositions that comprise one or more of the compounds described herein, or a pharmaceutically acceptable salt, tautomer, stereoisomer, deuterated analog, or solvate thereof, and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

Also provided herein, in some embodiments, are pharmaceutical compositions that comprise at least one compound described herein, at least one other therapeutic agent for treating or preventing a disease, disorder, or condition at least partially mediated by GPX4, and one or more pharmaceutically acceptable vehicles selected from carriers, adjuvants and excipients.

Also provided herein, in some embodiments, are pharmaceutical compositions that comprise at least one compound described herein, at least one other therapeutic agent for treating or preventing a neurodegenerative disease, and one or more pharmaceutically acceptable vehicles selected from carriers, adjuvants and excipients.

Also provided herein, in some embodiments, are pharmaceutical compositions that comprise at least one compound described herein, at least one other therapeutic agent for treating or preventing a disease associated with ferroptosis, and one or more pharmaceutically acceptable vehicles selected from carriers, adjuvants and excipients.

Also provided herein, in some embodiments, are pharmaceutical compositions that comprise at least one compound described herein, at least one other therapeutic agent for treating or preventing Sedaghatian-type Spondylometaphyseal Dysplasia (SSMD), and one or more pharmaceutically acceptable vehicles selected from carriers, adjuvants and excipients.

Also provided herein, in some embodiments, are pharmaceutical compositions that comprise at least one compound described herein, at least one other therapeutic agent for treating or preventing Ischemic Heart Disease, and one or more pharmaceutically acceptable vehicles selected from carriers, adjuvants and excipients.

Also provided herein, in some embodiments, are pharmaceutical compositions that comprise at least one compound described herein, at least one other therapeutic agent for treating or preventing male infertility, and one or more pharmaceutically acceptable vehicles selected from carriers, adjuvants and excipients.

The pharmaceutical compositions may be administered in either a single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant In certain embodiments, the pharmaceutical composition may be administered orally or intravenously.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained, or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, such as orally or nasally, from devices that deliver the formulation in an appropriate manner

### Kits

Provided herein are also kits that include a compound of the disclosure and suitable packaging. In one embodiment, a kit further includes instructions for use. In one aspect, a kit includes a compound described herein and a label and/or instructions for use of the compound in the treatment or prevention of an indication, including the disease, disorder, or condition described herein.

Provided herein are also articles of manufacture that include a compound described herein in a suitable container. The container may be a vial, jar, ampoule, preloaded syringe, and intravenous bag.

### Treatment Methods and Uses

Sedaghatian type Spondylometaphyseal Dysplasia (SSMD) is an autosomal recessive disease caused by variants in both alleles of the glutathione peroxidase 4 (GPX4) gene. Whole exome sequencing of a child affected with SSMD (and unaffected parents) revealed that mutations in GPX4 are likely responsible for SSMD. GPX4 is an antioxidant enzyme belonging to the family of mammalian isoenzymes called glutathione peroxidases produced by the gene GPX4. The GPX4 gene consists of seven exons and six introns and produces three isoforms of the protein—mitochondrial (mGPX4—UNIPROT P36969-1), cytosolic (cGPX4—UNIPROT P36969-2) and nuclear (nGPX4). All three isoforms are ubiquitously expressed in all tissues. Cytosolic and mitochondrial isoforms are known to be essential in the neurons of the developing brain and the nuclear isoform is predominantly synthesized during late spermatogenesis. It is a selenoprotein whose catalytic activity is indispensable for normal embryogenesis, maintaining mitochondrial oxidative phosphorylation, preventing lipid peroxidation, and plays a part in combating increased oxidative damage due to injury or chemotherapy.

mGPX4 has been shown to protect mitochondrial ATP generation against oxidative damage. Knockdown studies of GPX4 show reduction in expression of genes encoding components of Complex I, IV, and V. Overexpression of mGPX4 prevents release of the proapoptotic signaling molecule Cytochrome C from mitochondria and plays a key role as an anti-apoptotic agent in mitochondrial death pathways. mGPX4 protects cardiac contractile function and preserves electron transport chain activities following ischemia/reperfusion.

Cytosolic isoform of GPX4 is capable of reducing complex lipid peroxides such as those present in lipid membrane bilayer of cells. Polyunsaturated-fatty-acid-containing phospholipids (PL-PUFAs) are the lipids most susceptible to peroxidation with the bis-allylic carbons being most susceptible to attack by reactive oxygen species. GPX4 localizes to lipid membranes and reduces PUFA hydroperoxides using Glutathione (GSH) as substrate.

GPX4 deficiency is also associated with ferroptosis. Ferroptosis is a novel form of iron-dependent cell death. Loss of GPX4 results in peroxidation of the lipid cell membrane driven by an increase in reactive oxygen species (ROS). Depletion of the cell's intrinsic antioxidant Glutathione (GSH) also leads to ferroptosis. The presence of oxidizable phospholipids acylated with polyunsaturated fatty acid, presence of redox-active iron, and defective lipid peroxide repair are the hallmark features required for ferroptosis. α-Tocopherol (Vitamin E) works with GPX4 and GSH to stop lipid peroxidation, thereby halting ferroptosis.

Ferroptosis is emerging as a mechanism of cell death in various diseases, including cardiovascular diseases and acute kidney failure, and may also play a role in central degenerative brain disorders. Ferroptosis is driven by loss of activity of lipid repair enzyme GPX4 and subsequent accumulation of lipid hydroperoxides. Depletion of GPX4 in mice is known to induce ferroptotic cell death in embryo, testis, brain, liver, heart, and photoreceptor cells, cause rapid motor neuron degeneration and paralysis, promote cognitive impairment, trigger acute renal failure, and result in impaired T-cell-mediated immune response. Mice with depleted GPX4 showed hallmarks of ferroptosis including an increase in lipid peroxidation in various cell types.

GPX4 is further associated with ischemic heart disease and male infertility.

Provided herein are methods for treating or preventing a disease, disorder, or condition mediated, at least partially, by glutathione peroxidase 4 (GPX4) in a human patient in need thereof comprising administering a therapeutically effective amount of a compound as described herein, or a combination of the compounds described herein, or a composition as described herein. Also provided herein are methods for treating or preventing a disease, disorder, or condition associated at least partially with a deficiency in GPX4, comprising administering a therapeutically effective amount of a compound as described herein, or a combination of the compounds described herein, or a composition as described herein. Also provided herein are methods for upregulating the expression of GPX4, comprising administering a therapeutically effective amount of a compound as described herein, or a combination of the compounds described herein, or a composition as described herein.

Further provided herein are methods for treating or preventing SSMD comprising administering a therapeutically effective amount of a compound as described herein, or a combination of the compounds described herein, or a composition as described herein. Further provided herein are methods for treating or preventing neurodegenerative disease comprising administering a therapeutically effective amount of a compound as described herein, or a combination of the compounds described herein, or a composition as described herein. Further provided herein are methods for treating or preventing a disease, disorder, or condition at least partially associated with ferroptosis, comprising administering a therapeutically effective amount of a compound as described herein, or a combination of the compounds described herein, or a composition as described herein. Further provided herein are methods for treating or preventing ischemic heart disease comprising administering a therapeutically effective amount of a compound as described herein, or a combination of the compounds described herein, or a composition as described herein. Further provided herein are methods for treating or preventing male infertility comprising administering a therapeutically effective amount of a compound as described herein, or a combination of the compounds described herein, or a composition as described herein.

Some embodiments provide for a method for treating SSMD in a human patient in need thereof, comprising administering to the human patient an effective amount of azelnidipine or a pharmaceutically acceptable salt thereof, or a pharmaceutical composition comprising an effective amount of azelnidipine or a pharmaceutically acceptable salt thereof and a pharmaceutically acceptable carrier.

Some embodiments provide for a method for treating SSMD in a human patient in need thereof, comprising administering to the human patient an effective amount of bazedoxifene or a pharmaceutically acceptable salt thereof, or a pharmaceutical composition comprising an effective amount of bazedoxifene or a pharmaceutically acceptable salt thereof and a pharmaceutically acceptable carrier.

In any of the embodiments described herein, a patient is administered one or more of the compounds described herein. The one or more compounds can be administered simultaneously or sequentially.

In any of the embodiments described herein, the patient may be further administered a therapeutically effective amount of another therapeutic agent. The another therapeutic agent may be administered simultaneously or sequentially with a compound, or compounds, described herein or a composition described herein.

In any of the embodiments described herein, the patient may be further administered a therapeutically effective amount of another therapeutic agent useful for upgrading GPX4 expression. In any of the embodiments described herein, the patient may be further administered a therapeutically effective amount of another therapeutic agent useful for upregulating GPX4 expression or treating disease, disorder, or condition mediated, at least partially, by GPX4.

### Dosing

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered orally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day.

The compounds of the present disclosure or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In some embodiments, the method comprises administering to the subject about 1 mg to about 1000 mg per day of a compound described herein. In some embodiments, the method comprises administering to the subject about 1 mg to about 25 mg per day, such as about 8 mg to about 16 mg per day, of a compound described herein. In some embodiments, the method comprises administering to the subject about 5 mg to about 40 mg per day, such as about 10 mg to about 30 mg per day, of a compound described herein. In some embodiments, the method comprises administering to the subject about 8 mg to about 16 mg per day of azelnidipine. In some embodiments, the method comprises administering to the subject about 10 mg to about 30 mg per day of bazedoxifene.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

Compounds described herein were tested in three cell lines, RAG01 (derived from fibroblasts of a patient diagnosed with SSMD) fibroblasts, RAG02 (derived from fibroblasts of the patient's father), and GM09503 (from supplier Corriell) (derived from a healthy donor) to each generate 10-point concentration response curves (CRC). The three cell lines were each thawed seven (7) days prior to seeding. The RAG01 cells were thawed in a medium supplemented with a-tocopherol. Cells were passaged four (4) days prior to the seeding. The α-tocopherol was removed from the RAG01 cells. Cells were then seeded at 900 cells per well in 384-well plates. Two replicates were prepared for two independent (replicate) experiments. All plates were then incubated with compounds described herein for 72 hours, prior to CellTiter-Glo assay. The compounds were tested in 10-point concentration (CRCs) from 10 μM using 3.16×(half Log) dilutions. The assay was run using the CellTiter-Glo (Promega) following manufacturer's guidelines and luminescent spectrophotometric reading on PHERAstar FSX (BMG Labtech). A 0.1% dimethylsulfoxide (DMSO) was used as a blank (vehicle control); and a 1 μM Ferrostatin-1 was used as a control (CTRL).

The raw data was analyzed using Activity Base® software. The compounds were evaluated by efficacy (percentage effect) normalized to the vehicle control. The percentage effect (PE) was determined from the signal increase in comparison to the vehicle control according to the formula below:

$$P E = 100 - ( \frac{average signal vehicle control}{signal of compound} ) \times 100$$

The percentage effect values were then plotted against log concentration of the drug to generate a concentration response curve. The curves were fitted using a four-parameter fit, and curve tops and hillslopes were fixed where required and justified. The ECso values (from these curves) derived for individual compounds and obtained from the RAG01 cell line are shown in Tables 2A and 2B below.

TABLE 2A

| Ex. No. | EC₅₀ (μM) |
|---|---|
| 1 | 3.119 |
| 2 | 2.895 |
| 3 | 0.24 |
| 5 | 0.458 |
| 10 | 0.18 |
| 11 | 1.184 |
| 12 | 1.21 |
| 14 | 1.601 |
| 15 | 1.869 |
| 16 | 3.213 |
| 18 | 2.338 |
| 19 | 4.485 |
| 20 | 2.275 |
| 21 | 1.068 |
| 22 | 6.814 |
| 23 | 1.613 |
| 24 | 2.489 |
| 25 | 2.864 |
| 26 | 0.403 |
| 28 | 4.908 |
| 29 | 1.393 |
| 30 | 0.739 |
| 31 | 1.459 |
| 32 | 0.636 |
| 33 | 1.984 |
| 34 | 2.661 |
| 36 | 0.14 |
| 41 | 3.952 |
| 42 | 0.478 |
| 43 | 1.391 |
| 44 | 1.409 |
| 45 | 1.578 |
| 46 | 1.504 |
| 47 | 1.674 |
| 48 | 1.843 |
| 49 | 3.207 |
| 60 | 0.429 |
| 64 | 0.708 |
| 65 | 2.227 |
| 67 | 0.245 |
| 71 | 5.133 |
| 92 | 0.416 |
| 105 | 1.837 |

TABLE 2B

| Ex. No. | EC₅₀ (μM) |
|---|---|
| 4 | 0.43 |
| 6 | 1.626 |
| 7 | 1.664 |
| 8 | 5.51 |
| 9 | 0.053 |
| 13 | 1.573 |
| 17 | 7.362 |
| 27 | 3.107 |
| 35 | 2.567 |
| 37 | 2.149 |
| 38 | 4.307 |
| 39 | 0.652 |
| 40 | 2.735 |
| 50 | 0.928 |
| 51 | 1.353 |
| 52 | 2.38 |
| 53 | 2.711 |
| 54 | 0.381 |
| 55 | 1.321 |
| 56 | 1.408 |
| 57 | 2.412 |
| 58 | 0.35 |
| 59 | 0.928 |
| 61 | 0.652 |
| 62 | 3.847 |
| 63 | 5.447 |
| 66 | 0.202 |
| 68 | 0.366 |
| 69 | 2.384 |
| 70 | 0.485 |
| 72 | 0.077 |
| 73 | 0.168 |
| 74 | 0.764 |
| 75 | 0.377 |
| 76 | 0.48 |
| 77 | 0.611 |
| 78 | 0.654 |
| 79 | 0.559 |
| 80 | 0.452 |
| 81 | 0.688 |
| 82 | 0.916 |
| 83 | 1.043 |
| 84 | 1.192 |
| 85 | 1.244 |
| 86 | 1.46 |
| 87 | 1.65 |
| 88 | 2.296 |
| 89 | 2.672 |
| 90 | 0.117 |
| 91 | 0.268 |
| 93 | 0.438 |
| 94 | 0.652 |
| 95 | 0.739 |
| 96 | 0.956 |
| 97 | 1.101 |
| 98 | 1.148 |
| 99 | 1.265 |
| 100 | 1.306 |
| 101 | 1.376 |
| 102 | 1.409 |
| 103 | 1.56 |
| 104 | 1.724 |
| 106 | 2.47 |
| 107 | 3.554 |
| 108 | 3.773 |
| 109 | 3.936 |
| 110 | 5.56 |

Signal over baseline (S/B) values for RAG01 cells were robust and all greater than 4; and the Z scores (or Z′ values) for RAG01 cells were robust and higher than 0.6, indicating satisfactory assay quality. No effect of Ferrostain-1 was observed on either of RAG02 and GM09503 (control cell lines).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for treating a disease, disorder, or condition mediated by glutathione peroxidase 4 (GPX4) in a human patient in need thereof, comprising administering to the human patient an effective amount of a compound or a pharmaceutically acceptable salt thereof, wherein the compound is selected from the group consisting of a calcium channel blocker, GABA-ergic, antibiotic, radical trapping antioxidant (RTA), contraceptive, phytoestrogen antioxidant, antipsychotic, dopaminergic, serotonergic, iron chelator, opioid receptor antagonist, a δ receptor antagonist, neuromodulator, S1P₃ receptor antagonist, Cdc25 phosphatase inhibitor, antimalarial, PPARdelta antagonist, NAMPT activator, autophagy activator, neurogenesis activator, smoothened agonist, STAT3 inhibitor, antiviral, LDHA inhibitor, PDGFRbeta receptor inhibitor, NAT10 inhibitor, FXR agonist, CFTR potentiator, S1P₂ receptor antagonist, GPR139 agonist, HDAC class II inhibitor, multidrug resistance pump inhibitor, and gamma secretase inhibitor.

2. The method of claim 1, wherein the compound is a dihydropyridine calcium channel blocker or a pharmaceutically acceptable salt thereof.

3. The method of claim 1, wherein the compound is azelnidipine or a pharmaceutically acceptable salt thereof.

4. The method of claim 1, wherein the compound is bazedoxifene or a pharmaceutically acceptable salt thereof.

5. The method of claim 1, wherein the compound is an estrogen receptor modulator.

6. The method of claim 1, wherein the disease, disorder, or condition is associated with a deficiency in GPX4.

7. The method of claim 1, wherein the disease, disorder, or condition is Sedaghatian-type Spondylometaphyseal Dysplasia (SSMD).

8. (canceled)

9. (canceled)

10. The method of claim 1, wherein the compound is GABA_{B} modulator.

11. (canceled)

12. The method of claim 1, wherein the compound is cryptic RTA.

13. The method of claim 12, wherein the cryptic RTA is selected from the group consisting of is selective estrogen receptor modulators (SERM) beta-adrenergic blocker, beta-adrenergic agonist, alpha-adrenergic antagonist, alpha-adrenergic agonist.

14. (canceled)

15. (canceled)

16. (canceled)

17. (canceled)

18. (canceled)

19. (canceled)

20. (canceled)

21. The method of claim 1, wherein the compound is RTA, wherein the RTA is anti-inflammatory.

22. The method of claim 21, wherein the anti-inflammatory is LOX/COX inhibitor, leukotriene production blocker, antioxidant, or 5-lipoxygenase inhibitor.

23. (canceled)

24. The method of claim 1, wherein the compound is phenothiazine, EGFR inhibitor, EGFR and ErbB2 inhibitor, or kinase inhibitor.

25. The method of claim 24, wherein the kinase inhibitor is Tpl2/MAP3K8 inhibitor, PKCPII inhibitor, PKC inhibitor, mixed kinase inhibitor, ALK inhibitor, FAK inhibitor, Ackl/ALK inhibitor, GSK-3 inhibitor, MPS1 kinase inhibitor, Mpsl inhibitor, Tyrosine kinase inhibitor, p38 inhibitor, GSK-3/CDK inhibitor, PIP5K1C inhibitor, angiokinase inhibitor, RTK inhibitor, HER1/2 kinase inhibitor, or PNKP inhibitor.

26. The method of claim 1, wherein the compound is antiarrhythmic, antispasmodic, or antipruritc.

27. (canceled)

28. (canceled)

29. (canceled)

30. The method of claim 1, wherein the compound is antipsychotic, wherein the antipsychotic is a 5-HT_{2A} antagonist or a dopamine receptor agonist.

31. (canceled)

32. The method of claim 1, wherein the compound is dopaminergic, wherein the dopaminergic is D₁D₅ partial agonist, D₁ receptor agonist, or D₃ agonist.

33. (canceled)

34. The method of claim 1, wherein the compound is serotonergic, wherein the serotonergic is 5-HT_{1A} agonist, 5-HT₆ partial agonist, or 5-HT_{2b} antagonist.

35. (canceled)

36. (canceled)

37. The method of claim 1, wherein the compound is neuromodulator, wherein the neuromodulator is sigma receptor agonist, NMDA antagonist, triple monoamine reuptake inhibitor, or NPY Y₅ antagonist.

38. A method for treating a disease, disorder, or condition mediated by glutathione peroxidase 4 (GPX4) in a human patient in need thereof, comprising administering to the human patient an effective amount of a compound of Table 1A or 1B, or a pharmaceutically acceptable salt thereof.
